(19) United States
(12) Patent Application Publication
(10) Pub. No.: US 2025/0037862 A1
(43) Pub. Date: Jan. 30, 2025

(54) Diagnostic Method and System

(71) Applicant: Limbic Limited, London (GB)

(72) Inventors: Ross Edward Francis Harper, London (GB); Max Rollwage, Essex (GB)

(21) Appl. No.: 18/710,169

(22) PCT Filed: Nov. 15, 2022
(86) PCT No.: PCT/GB2022/052898

§ 371 Date: May 14, 2024

(30) Foreign Application Priority Data
Nov. 15, 2021 (GB) 2116425.6

(51) Int. Cl. G16H 50/20; G16H 40/60

(52) U.S. Cl. CPC G16H 50/20; G16H 40/60

(57) ABSTRACT

A method for performing automated diagnostics, comprising processing first input data relating to a speech or text input signal to generate a representation of the first input data and generating a first output based at least in part on the representation of the input data; determining a preliminary diagnosis output comprising at least one preliminary diagnosis of the problem by processing, using a preliminary diagnosis machine learning model, the first output; determining, at the one or more processors and based at least in part on the preliminary diagnosis output, at least one dialogue system output; outputting, by way of an output of the diagnostics system, the dialogue system output.

### FIELD

The present invention relates to a computer-implemented method, system and a computer software product for performing diagnostics and for generating dynamic dialogue system output for automatic diagnostics.

### BACKGROUND

Diagnostics within complex systems is integral to many domains such as technical support, horticulture, medicine (both physical health and mental health), construction and industry. Performing diagnostics on complex systems often rely on one or more sets of predetermined questions that have been configured to accurately identify a problem from a range of possible problems. For example, accurate diagnosis of a problem with technical hardware (such as computers or industrial machinery) may require a user of the hardware to answer a number of predetermined questions or sets of predetermined questions relating to the symptoms of the problem. Similarly, diagnosis of physical or mental health conditions may require the administration of multiple questions or sets of questions relating to the patient's symptoms. Given the size and complexity of some complex systems, the number of possible questions can be extensive. Determining the particular questions (or sets of questions) to ensure sufficient coverage of the problem domain and the order in which to ask those questions may not be straightforward. Additionally, existing systems may require users to answer a large number of questions causing users (or operatives administering the questions to a user) to avoid one or more questions necessary to accurately diagnose a problem. Furthermore, overlapping sets of questions can lead to duplication of questions.

### SUMMARY OF INVENTION

There is described herein a computer-implemented method for automated diagnostics. The method comprises receiving, at an input of a diagnostics system, input data relating to a speech or text input signal originating from a user device, the first input data indicating at least one problem; processing, at one or more processors executing a first input pre-processing module comprising a first input pre-processing machine learning model, the first input data to generate a representation of the first input data and to generate a first input pre-processing module output based at least in part on the representation of the first input data; processing, at the one or more processors, the first input pre-processing module output using a preliminary diagnosis machine learning model to determine a preliminary diagnosis output comprising at least one preliminary diagnosis of the problem; determining, at the one or more processors and based at least in part on the preliminary diagnosis output, at least one dialogue system output; outputting, by way of an output of the diagnostics system, the dialogue system output; receiving, at the input of the diagnostics system, additional input data responsive to the dialogue system output; processing, at the one or more processors, the additional input data to determine one or more further diagnoses; and outputting, by the output of the diagnostics system, an indication of the one or more further diagnoses.

In this way, the method enables a dialogue system output to be determined based upon a preliminary diagnosis generated based upon input text or speech data and for a further diagnosis to be generated in response to processing responses to the dialogue system output. The input data may comprise free text or free speech. The dialogue system output may be one or more queries or questions, such as questions that must be asked to diagnose the problem.

The first input pre-processing machine learning model may comprise one or more feed-forward neural networks.

Second input data may be received at the input. It will be appreciated that the input may include any or multiple means of input to the dialogue system. The second input data may comprise a plurality of answers responsive to predetermined questions output by the diagnostics system. The second input data may be processed at a second input pre-processing module comprising a second input pre-processing machine learning model to generate a second input pre-processing module output, the second input pre-processing module output comprising a prediction of at least one problem based at least in part upon the second input pre-processing module output. Determining the preliminary diagnosis output may comprise processing the second input pre-processing module output at the preliminary diagnosis machine learning model and the preliminary diagnosis output may be based at least in part on the second input pre-processing module output.

In this way, the preliminary diagnosis may be based upon multiple inputs with different data modalities, each processed by an input pre-processing module adapted for that data modality. The method may also include outputting the predetermined questions using the output of the dialogue system.

Third input data may be received from one or more sensors, the third input data comprising a plurality of sensor signals measuring a characteristic of a user. The third input data may be processed at a third input pre-processing module configured to generate a third input pre-processing module output comprising one or more principal components of the third input data. Determining the preliminary diagnosis output may comprise processing the third input pre-processing module output at the preliminary diagnosis machine learning model and the preliminary diagnosis machine learning model may be configured to determine the preliminary diagnosis output based at least in part on the third input pre-processing module output. The third input data may include, for example, response times, but more generally may include any sensor data as is described in more detail herein.

Fourth input data may be received from one or more sensors, the fourth input data comprising a plurality of sensor signals measuring a response time of a user when answering each of a plurality of questions output by the dialogue system. The fourth input data may be processed at a fourth input pre-processing module configured to generate a fourth input pre-processing module output comprising at least one of: an average response time, variation between one or more response times, a minimum response time and a maximum response time. Determining the preliminary diagnosis output may comprise processing the fourth input pre-processing module output at the preliminary diagnosis machine learning model and the preliminary diagnosis machine learning model is configured to determine the preliminary diagnosis output based at least in part on the fourth input pre-processing module output.

Determining one or more further diagnoses of the problem may comprise providing the fifth input data to a machine learning classifier trained to determine the one or more further diagnoses of the problem based upon the fifth input data.

As action may be caused to be taken or scheduled, responsive to the one or more further diagnoses. A priority may be determined based upon the one or more further diagnoses, and the action may be determined responsive to the priority. The action may comprise, by way of example, at least one of: allocating a user of the user device to a treatment pathway for treatment by a clinician; scheduling an appointment with a clinician; establishing a communication channel with an emergency service; and generate and/or output one or more instructions and/or treatment plan actions for the user.

The preliminary diagnosis machine learning model may comprise a gradient boosting decision tree classifier.

The preliminary diagnosis model may have been trained using a multi-class objective function, such as a soft probability objective function.

The objective function may have been defined by a combination of a micro averaged accuracy score and a macro averaged accuracy score, wherein the micro averaged accuracy score was defined by an overall accuracy diagnoses output by the preliminary diagnosis model independent of an accuracy of individual diagnosis categories and the macro averaged accuracy score were defined by accuracies of individual diagnosis categories output by the preliminary diagnosis model and averaged with equal weight.

The first input pre-processing module may comprise a plurality of first input pre-processing machine learning models each configured to generate a respective representation of the first input data having a lower dimensionality than the first input data and each trained on a different dataset. The method may comprise generating the first input pre-processing module output based at least in part on the plurality of representations of the first input data.

In this way, the input data may be processed by a number of models, and each can be configured to provide a different output based on the input data based on the dataset on which it was trained.

The first input pre-processing module may comprise at least one embedding machine learning model configured to generate an embedding of the first input and to provide the embedding as an input to the first input pre-processing machine learning model.

The first input pre-processing module may comprise a classifier machine learning model configured to determine, based on the first input data, one or more categories of problem indicated in the first input data.

The preliminary diagnosis model may be configured to determine a respective probability value for each of a plurality of categories. The method may further comprise determining one or more of the plurality of categories based on the respective probability values; and determining the at least one dialogue system output by determining at least one dialogue system output associated with each of the determined one or more of the plurality of categories.

Determining one or more of the plurality of categories may comprise selecting a minimum number of the plurality of categories having a cumulative probability that exceeds a cumulative probability threshold.

At least a part of the first input pre-processing module may be operated on a client device, and the preliminary diagnosis model may be operated on a server device. The method may therefore include transmitting processing the input data at the client device and transmitting the processed input data to the server device to perform the preliminary diagnosis.

The input data may be one of a plurality of user inputs, each having a different data modality. The method may further comprise providing respective ones of the plurality of user inputs to respective input pre-processing modules, each input pre-processing module configured to generate a respective input pre-processing module output for inputting to the preliminary diagnosis model. Determining the preliminary diagnosis output may comprise processing each of the respective input pre-processing module outputs at the preliminary diagnosis machine learning model to provide the preliminary diagnosis output based at least in part on each of the respective input pre-processing module outputs.

The input data may relate to mental health. The preliminary diagnosis output may comprise at least one diagnosis of one or more mental health conditions. The one or more dialogue system outputs may comprise questions for confirming or disconfirming the at least one diagnosis of one or more mental health conditions.

Determining at least one dialogue system output may further comprise selecting one or more sets of questions relating to the at least one preliminary diagnosis and may comprise de-duplicating questions present in more than one of the one or more sets of questions relating to the at least one preliminary diagnosis.

There is also described herein one or more computer readable media, storing computer readable instructions configured to cause one or more processors to perform any of the methods described herein.

There is also described herein a diagnostics system, comprising one or more processors; and one or more computer readable media configured to cause the one or more processors to perform any of the methods described herein.

### BRIEF DESCRIPTION OF DRAWINGS

Embodiments will now be described, by way of example only and with reference to the accompanying drawings having like-reference numerals, in which:

FIG. 1a shows a schematic illustration of a system suitable for implementing one or more embodiments;

FIG. 1b is a schematic illustration of an example arrangement of components that may be used in one or more devices of the system of FIG. 1;

FIG. 2 shows an illustration of a dialogue system interface according to an example method described herein;

FIG. 3a shows a schematic illustration of a system for implementing techniques described herein;

FIG. 3b shows a flowchart of an example method that may be performed by the system of FIG. 3;

FIG. 4 shows an illustration of a text or audio pre-processing model for inclusion in the system of FIG. 3a;

FIG. 5 shows an illustration of a question pre-processing model for inclusion in the system of FIG. 3a;

FIG. 6 shows an illustration of a response time pre-processing model that may form part of the system of FIG. 3a;

FIG. 7 shows an illustration of an action logic for processing outputs from the system of FIG. 3a;

FIG. 8 shows an overview of an example operation of a triage system;

FIG. 9 shows an example sequence of steps performed by a triage system;

FIGS. 10-13 shows flow diagrams of example processes for processing inputs;

FIG. 14 shows a flow diagram of an example process for processing outputs from one or more of the processes of FIGS. 10-13; and

FIG. 15 shows a comparison of a test system against human experts.

### SPECIFIC DESCRIPTION

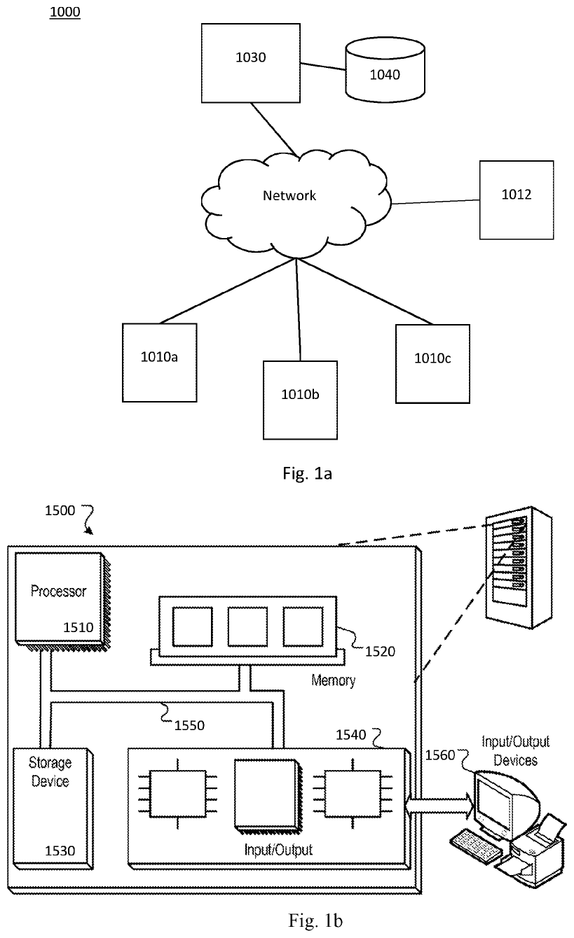

Referring to FIGS. 1 to 6, the details of one or more aspects of methods, systems and a computer software product for automatically generating user interface output for diagnostics will now be described in more detail below. The use of the same reference numbers in different instances in the description and the figures may indicate like elements:

Referring to FIG. 1a, there is shown a computer system 1000 suitable for implementing parts of the methods described herein. In the system 1000, user devices 1010a-c (collectively referred to as user devices 1010) are configured to communicate over a network with a server 1030. The server has access to storage 1040. For example, the storage 1040 may be local to the server 1030 (as depicted in FIG. 1) or may be remote. While the storage is depicted as a single storage 1040, it will be appreciated that the storage 1040 may be distributed across a plurality of devices and/or locations. The server 1030 is configured to make available over the network one or more applications for use by user devices 1010. In particular, the server 1030 is configured to make available a diagnostic application for assisting users of the user devices 1010 in performing a diagnostic. The diagnostic application may provide a dialogue system (which may be, e.g., a chatbot) that receives inputs from the user and processes the inputs to generate appropriate outputs. The diagnostic application may be accessed by a user device 1010 through, for example, a web-browser or a client application operating locally on the user device 1010. The storage 1040 may store data (e.g. in one or more databases) used by the application. For example, the storage 1040 may store sets of questions to ask a user of the application and may store answers provided by the user in response to those questions. The storage 1040 may further store machine-learning models used by the application to process users' answers. The storage 1040 may further store individual profiles and credentials for respective users of the application so that a user's answers may be uniquely and securely identified with that user. The server 1030 and/or the user devices 1012 may be in further communication with one or more third party devices 1012. The diagnostic application may transmit, from a user device 1010 or the server 1030, information generated during the diagnostic to the one or more third party devices 1012, or may automatically communicate with third party devices 1012 to cause services to be scheduled or provided by third parties associated with the third party devices 1012.

Each of the user devices 1010 may be any device that is capable of accessing the application provided by the server 1030. For example, the user devices may include a tablet computer, a desktop computer, a laptop, computer, a smartphone, a wearable device or a voice assistant.

The application provided by the server 1030 provides an interface to output information to a user and to enable a user to input information. For example, the interface may include a textual interface in which the user inputs text (e.g. using a keyboard or handwriting recognition interface associated with the user device 1010) and the application provides outputs in a text format (e.g. using a display associated with the user device 1010). Alternatively or additionally, the interface may include an audio interface in which the user inputs audio (e.g. using a microphone associated with the user device 1010) and the application provides outputs in an audio format (e.g. using a speaker associated with the user device 1010). It will be appreciated that the interface may include a plurality of input/output modalities including text, audio, video, animation, etc. Additionally, it will be appreciated that inputs and outputs provided in a first format may be converted to a second format. For example, where the application provides an audio input interface, audio inputs provided by the user may be converted to a textual format by the application for further processing. Similarly, where the application provides an audio output interface, audio outputs may be generated by converting textual outputs to an audio format.

Referring to FIG. 1b, there is shown an example computer system 1500 that may be used to implement one or more of the user devices 1010, the server 1040 and the third party devices 1012. The methods, models, logic, etc., described herein may be implemented on a computer system, such as the computer system 1500. The computer system 1500 may comprise a processor 1510, memory 1520, one or more storage devices 1530, an input/output processor 1540, circuitry to connect the components 1550 and one or more input/output devices 1560. While schematic examples of the components 1510-1550 are depicted in FIG. 1b, it is to be understood that the particular form of the components may differ from those depicted as described in more detail herein and as will be readily apparent to the skilled person.

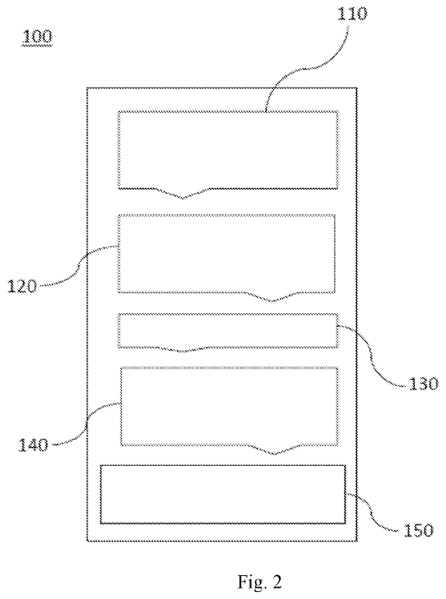

Referring to FIG. 2, there is shown an example user interface for the application provided by the server 1030. In the example of FIG. 2, the user interface takes the form of a chat interface 100. The chat interface 100 is presented on a display associated with a user device 1010.

The chat interface 100 may present one or more initial questions 110 to a user, to which the user will submit a first response 120. This first response 120 may be in the form of free text, or may involve the selection of one or more options from a list of answers, or a combination of these. Based on the first response 120, the chat interface 100 can present one or more follow up questions 130 to the user, to which one or more further responses 140 can be provided by the user. The one or more follow up questions may be determined by a machine learning model processing the first response to determine a preliminary diagnosis. One or more rounds of questions and responses may be provided such that a machine learning model makes multiple, iterative preliminary diagnoses and elicits more information from the user before determining a final diagnosis. Typically, when using a smartphone to display the chat interface 100, a text input area 150 such as a keyboard or handwriting recognition area of a screen will typically be present on the user interface.

As described above, it will be appreciated that user interfaces other than chat interfaces may be provided. The user interface provides a front-end of the application. Other components of the application includes communication interfaces to enable communication with the user devices 1010 and application logic configured to assist the user of a user device 1010 in performing a diagnostic. For example, the application logic may include one or more machine-learning models configured (e.g. trained) to process input data provided by the user to generate one or more outputs that facilitate the user in performing the diagnostic.

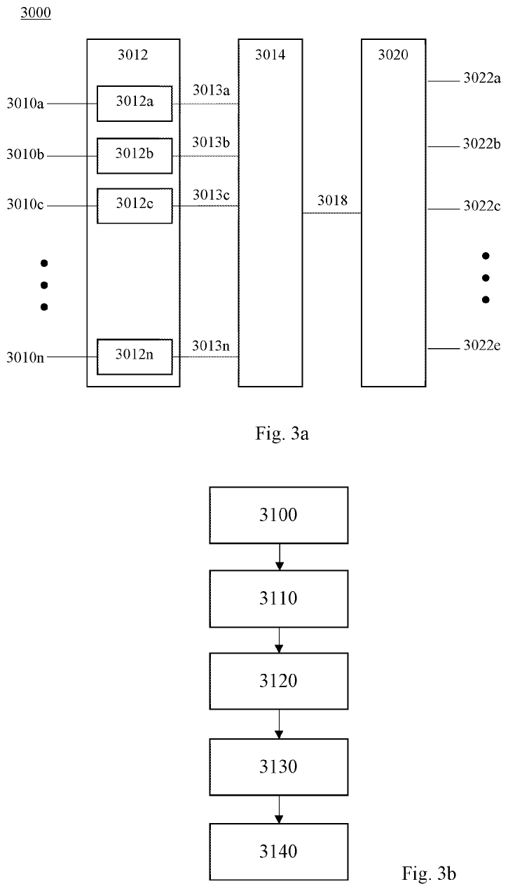

Referring to FIG. 3a, there is shown a schematic overview of a system 3000 for processing data received from a user using the user interface and for generating outputs to provide to the user in the user interface for facilitating a diagnostic.

In FIG. 3a, the system 3000 a plurality user inputs 3010a-3010n (collectively inputs 3010) are provided to an input pre-processing stage 3012. One or more of the inputs 3010 may be inputs provided by the user in response to a question provided by the user interface of the application. For example, one or more of the inputs 3010 may be inputs provided by the user to represent the characteristics of the problem to be diagnosed. For example, one or more of the inputs 3010 may take the form of a description of the problem. One or more of the inputs 3010 may include answers to specific questions output to the user through the user interface of the application. One or more of the inputs 3010 may therefore take the form of a selection of a predetermined answer to a predetermined question.

One or more of the inputs 3010 may be measurements made by the user or on the user. For example, the inputs 3010 may include physiological characteristics of the user measured by sensors associated with (e.g. part of or in communication with) the user device 1010, such as heart rate (e.g. using a heart rate monitor), blood pressure (e.g. measured using a blood pressure sensor), oxygen saturation (e.g. using a pulse oximetry sensor), galvanic skin response (e.g. using a galvanic skin response sensor), electrocardiography, photoplethysmography or other. The inputs 3010 may include other inputs determined by the user device, such as location of the user (e.g. using a GPS sensor, WiFi measurements, etc), accelerometery, video, audio, temperature, light intensity, touch screen events, cursor movements, haptic feedback, type of user device, or other.

The input pre-processing stage 3012 comprises a plurality of input pre-processors 3012a-3012n. While a plurality of input pre-processing models are shown in FIG. 3a, in other example implementations, the input pre-processing stage 3012 may include a single input pre-processing model. Further, while the example of FIG. 3a depicts a one-to-one relationship between the inputs 3010a-3010n and the input pre-processors 3012a-3012n, this is merely exemplary. In other example implementations, one or more of the input pre-processors 3012a-3012n may receive the same user input.

Each input pre-processing model of the input pre-processing stage 3012 is configured to process a received input in order to generate an output 3013a-3013n (collectively 3013) for processing by a preliminary diagnostics model 3014. The preliminary diagnostics model 3014 is configured to receive inputs from the input pre-processing stage 3012 and to process the received inputs to make an preliminary diagnostic of one or more likely problems and provide an output 3018 representing the determined one or more likely problems. The output 3018 is provided as an input to an output generator 3020. The output generator 3020 is configured to determine, based on the output 3018, one or more questions 3022a-3022n to present to the user to further the diagnostic. The determined one or more questions 3022a-3022n may be provided to the user through the user interface. Inputs may be transmitted from the client device to the server as they are received at the client device or may be transmitted once all of the inputs have been received. The inputs may be processed by each of the input pre-processing modules as respective inputs are received and the outputs of the input pre-processing modules may be stored at the server until all of the inputs have been processed.

As described above, the input pre-processing stage 3012 includes one or more input pre-processing models 3012. A number of example input pre-processing models are now described.

Referring now to FIG. 3b, there is shown an example method that may be performed, for example by the system of FIG. 1 implementing the architecture of FIG. 3a, to generate a dialogue system output. At a step 3100, first input data is received at an input. The first input data indicates at least one problem. For example, the first input data may be one or more of the inputs 3010. At step 3110, the input data is processed at a first input pre-processing module comprising a first input pre-processing machine learning model. For example, the first input pre-processing module may be one of the input pre-processing modules of the input pre-processing stage 3012. The first input pre-processing machine learning model is configured to generate a representation of the first input data and the first input pre-processing module is configured to generate a first input pre-processing module output based at least in part on the representation of the first input data. For example, the first input pre-processing module output may be one of the outputs 3013.

At step 3120, a preliminary diagnoses output is determined by processing the first input pre-processing module output at a preliminary diagnoses machine learning model configured to determine the preliminary diagnoses output based at least in part on the first input pre-processing module output. For example, the preliminary diagnosis machine learning model may be the preliminary diagnosis model 3014. The preliminary diagnoses output comprises at least one preliminary diagnoses of the problem. For example, the preliminary diagnosis output may be the output 3018.

At step 3130, at least one dialogue system output is determined based at least in part on the preliminary diagnosis output. For example, the at least one dialogue system output may be determined by the output generator 3020. The dialogue system output may be one or more of the outputs 3022. At step 3140, the dialogue system output is output by way of an output of the system.

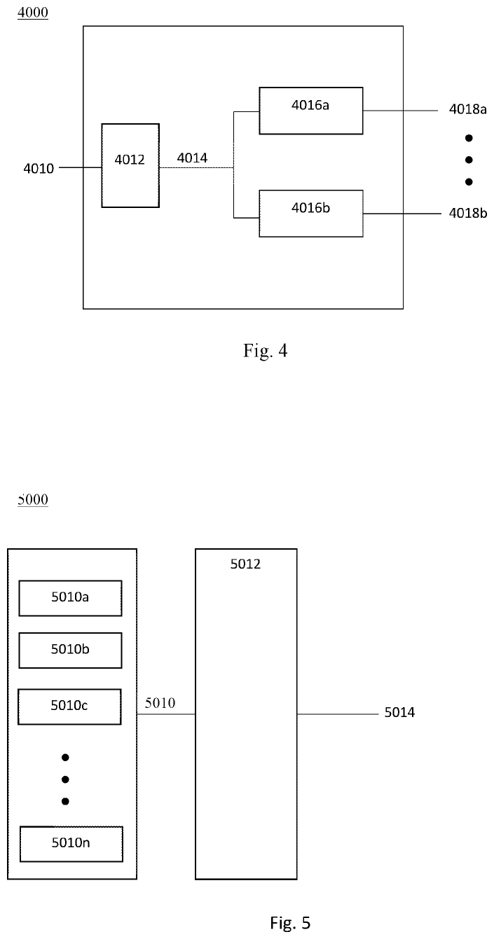

Referring to FIG. 4, there is shown an input pre-processor 4000 configured to classify a text and/or audio input 4010 to an output 4018 indicating one or more possible problems characterised by the input 4010. The inputs 4010 may be one of the inputs 3010. The output 4018 may be one of the outputs 3013. FIG. 4 represents one example system, though it is to be understood that the classification may use any appropriate classification methods as will be apparent to the skilled person.

The input 4010 may include text and/or audio input. For example, the input 4010 may be a text or audio input from a user of a user device 1010 and may characterise the problem that is to be diagnosed. The input 4010 may include a video input and an audio input may be obtained from the video input using standard video processing techniques. Similarly, a text input may be obtained from an audio or a video input (e.g. using a standard speech to text techniques such as a speech-to-text machine-leaned model). The input 4010 may be received in response to a prompt provided by the application through the user interface. For example, the prompt may ask the user to describe the problem in their own words. It will be appreciated that, in view of the free nature of the responses provided by a user, the input 4010 may have extremely high dimensionality.

The input pre-processor 4000 includes an embedding model 4012 configured to receive as input the input 4010 and to process the input 4010 to transform at least a part of the input into embedding 4014. The embedding model 4012 may be audio and/or text embedding models depending upon the nature of the input 4010. The embedding model 4012 may include any appropriate embedding model. For example, the model 4012 may include a BERT (Bidirectional Encoder Representations from Transformers) model, a sentence-BERT (s-BERT) model, Doc2VEC, InferSent and/or Universal Sentence Encoder models as will be appreciated by those skilled in the art. It will further be appreciated that the models 4012 may comprise other layers in addition to an embedding model layer, such as a pooling layer. The embedding model may be a pre-trained embedding model. The embedding model may be pre-trained on the domain of interest, that is the domain of the problem to be diagnosed. For example, the embedding model may be trained from the outset (i.e. “from scratch”) on a dataset from the domain of interest, or may be pre-trained on a broader dataset and may be further trained on a dataset from the domain of interest. By training, or further training, the embedding model on the domain of interest, the embedding model may be able to generate more accurate embeddings of the inputs 2010. Alternatively, the embedding model may be a general embedding model without any specific training on a dataset from the domain of interest.

By processing the input 4010 to generate the embedding 4014, the potentially high-dimensional input 4010 is reduced to an input with lower dimensionality while retaining the meaning of the input 4010. In this way, the input 4010 may be more efficiently stored and processed in subsequent stages of the diagnostic. In some example implementations, the entire input pre-processor 4000, or a part of the input pre-processor 4000 including the embedding model 4012, may execute on a user device 1010. In this way, a reduction in the amount of data that is sent over the network may be achieved.

The input pre-processing model may comprise more than one embedding model. For example, in an example, a further embedding may be generating using an n-gram bag of word embedding model (e.g. using 3-grams). A plurality of embeddings may be combined (e.g. concatenated or otherwise combined) for each input 4010.

The embedding (or combined embedding) 4012 is provided to one or more classifiers 4016. While two classifiers 4016a, 4016b are depicted in FIG. 4, it will be understood that the input module 4000 may include only one classifier or may include more than two classifiers. While in the example of FIG. 4, it is the embedding 4012 that is provided as input to the classifiers 4016, in other example implementations, the embedding model may be omitted such that the input 4010 is provided directly to the classifiers 4016.

The one or more classifiers 4016 may be pre-trained machine learning classifiers, trained on a training set of data relevant to the problem domain of the diagnostic. In this way, the classifiers 4012 may be configured to provide a further reduction in the dimensionality of the input 4010. That is, by pre-training the classifiers 4016 on the problem domain, the classifiers can be trained to identify categories (which may be referred to as problem descriptors) of problems described in the input 4010. The high-dimensional input 4010 may therefore be reduced to a very dense representation comprising a predefined list of possible classes of problem. The classifiers 4016a may use any appropriate techniques. By way of example only, the classifier may use, feedforward neural networks, Gradient Boosting Decision Trees, support vector machines (SVMs) or a Bayesian classifier (for example using a Naïve Bayes algorithm). In one example, a first classifier 4016a may comprise a feedforward neural network with two hidden layers, and a second classifier 4016b may comprise a feedforward network with a single hidden layer. The feedforward networks may be trained in accordance with any appropriate training technique such as backpropagation. Any appropriate objective function may be used. For example, a multi-class objective may be used. For example, a multi-class softmax objective may be used to output a multi-class classification using the softmax objective. Alternatively, a multiclass “softprob” (soft probability) objective may be used to output a multi-class classification including a probability of the input belonging to each class. Any appropriate loss function may be used. For example, a multi-class log loss function (or cross-entropy loss) may be used.

The classifiers 4016 may be trained using training data based on an existing corpus of user descriptions of problems together with ground truth diagnoses of the problem. In one example implementation, the training data may be obtained from conversations between users or between users and experts, for example from phone calls, forum posts or one-to-one support. In one example implementation, a title/heading of a forum post may be used as a ground truth label. The embedding model 4012 may generate the embedding 4014 from the text of the body of the post. The classifiers 4016 may be trained to identify the title/heading of the post from the embedding of the text in the body of the post.

Where more than one classifier 4016a is provided, each classifier 4016a, 4016b may be trained using a different training set of data. For example, a first training set may include data representing discussions between users. Such a training set may be obtained from, for example, a user forum. A second training set may include discussions between users and experts. Such a training set may be obtained from, for example, expert support systems.

Where more than one classifier 4016 is provided, each classifier may have the same architecture or a different architecture. By providing a plurality of classifiers 4016 with differing architecture and/or trained on different data sets, the input pre-processor 4000 may be able to capture different representations of problems provided in inputs 4010 and more accurately support further diagnosis. It will be appreciated that each classifier 4016 may be configured to output differing classifications depending on the coverage of the respective training sets on which they are trained. For example, a first training set may be concerned only with problem classes A, B, C while the second training set may be concerned only with problem classes A, B, X. In this cases, the classifiers trained on the respective training sets will output different labels 4018. Each of the classifiers 4018 may be multi-class classifiers or single class classifiers such that the one or more outputs 4018 may each include a single class or a list of possible classes. The outputs 4018 may comprise an indication of a probability of the determined class(es) (or a confidence score or value). One or more of the outputs may 4018 may further comprise a confidence score associated with the probability, for example, if one or more of the classifiers is a Bayesian classifier.

Referring to FIG. 5, there is shown an input pre-processor 5000 configured to classify input 5010 comprising responses to one or more predetermined questions provided to the user through the user interface. The input pre-processor 5000 may operate on either the user device or the server 1030. FIG. 5 represents one example system for classifying predetermined questions, though it is to be understood that the classification may use any appropriate classification methods as will be apparent to the skilled person. Predetermined questions or questionnaires may be provided to a user during an input stage (i.e. prior to selection of questions by the output generator 3020) where it is necessary to ask one or more questions from the set possible questions. That is, one or more questions may be asked during the input stage when those questions are mandatory such that it would be necessary to ask those questions at some stage of the diagnostic. Predetermined questions may also be provided in the input stage where a subset of one or more questions provide a predetermined coverage of most likely diagnostics. For example, where the diagnostic method is diagnosing a problem with a computing device, and where a “memory seating issue” is the most commonly diagnosed problem, questions relating to memory seating may be asked during the input stage.

The input pre-processor 5000 receives an input 5010 comprising one or more answers 5010a-5010n to one or more predetermined questions. For example, referring to FIG. 1, the storage 140 may store a plurality of questions that can be provided to the user through the user interface. The application may be configured to ask a predetermined one or more of these questions through the interface and to provide answers received from the user to the input pre-processor 5000. The answers 5010 may be free text answers. Alternatively, the answers 5010 may be selected from a set list of possible valid answers for the question. The user interface may provide the set list of possible answers for the user to select from. For example, one or more of the predetermined questions may have binary (e.g. “yes”, “no”) answers. One or more of the predetermined questions may have answers selected from a scale (e.g. 1 to 5 or 1 to 10).

The input 5010 is provided to a classifier 5012 configured (i.e. trained) to determine a problem class from the input 5010 and to provide an output 5014 representing the problem class. The classifier 5012 may be a pre-trained machine learning classifier, trained on a training set of data relevant to the problem domain of the diagnostic. The classifier 5010 may use any appropriate classification techniques. In one advantageous example, the classifier may use a Gradient Boosting algorithm with decision trees as the weak learners, but may use any other appropriate classifier such as Bayesian classifiers.

It has been found that for inputs 5010 comprising answers to questions for which there are a set number of predetermined valid answers, a Gradient Boosting decision tree classifier provides a particularly efficient implementation, thereby reducing both processing time and processing resources need to pre-process the input 5010, while providing an accurate cla output 5014. The gradient boosting classifier algorithm may be, for example, an XGBoost algorithm. A regularised gradient boosting algorithm such as XGBoost may be particularly beneficial to enable parallelization of processing decisions within the decision tree, allowing for the question answers to be processed more quickly and with more efficient use of processing resources. In other examples, the classifier may use gradient boosting techniques such as CatBoost, LightGMB or others as will be known to those skilled in the art.

In an example in which the classifier uses an XGBoost Gradient Boosting algorithm with decision trees as a weak learners, an example implementation may use the following hyperparameters:
Learning rate (eta): 0.01
Maximal depth of trees (max_depth): 10
Maximum number of estimator trees: 5000
Gamma: 5
Alpha: 1
Subsample ratio of columns by level (Colsample by level): 5

The maximal depth of trees indicates the maximal depth of any decision tree, the maximum number of estimator trees indicates the maximum number of trees, the learning rate indicates the step size shrinkage used in updates to prevent overfitting—after each boosting step, the weights of new features are obtained and the learning rate shrinks the feature weights to make the boosting process more conservative. Gamma indicates a minimum loss reduction required to make a further partition on a leaf node of the decision tree—larger gamma values cause the gradient boosting model more conservative. Alpha is an L1 regularization term on weights-increasing values of alpha will make model more conservative. Subsample ratio of columns by level indicates the subsample ratio of columns for each level of the tree.

The classifier 5012 may be trained using any appropriate loss function. For example, where the classifier 5012 is a gradient boosting classifier, the loss function may be a multi-class log loss function. Training data may comprise a corpus of existing user answers to the predetermined questions, together with actual diagnoses associated with those answers providing a ground truth label. The classifier 5012 may be multi-class classifier or a single class classifier such that the output 5014 may include a single class or a list of possible classes. The outputs 5014 may comprise an indication of a probability of the determined class(es) and may further include a confidence score.

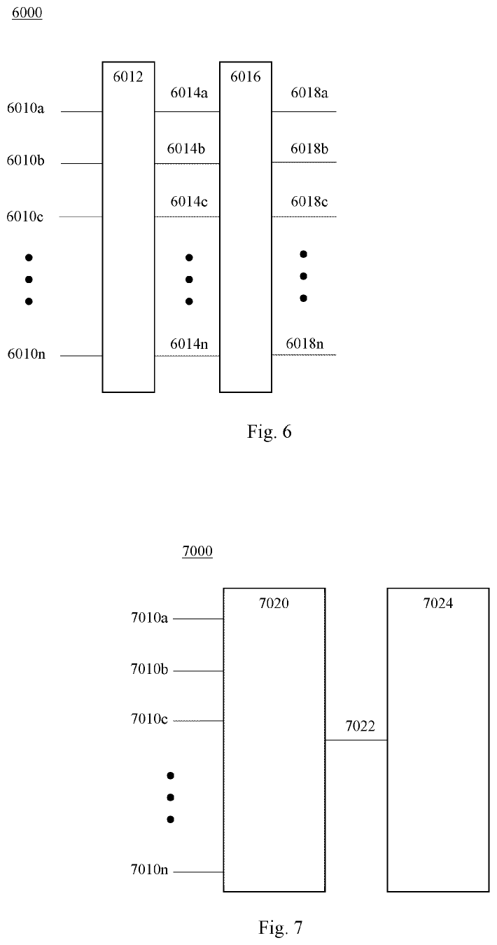

Referring to FIG. 6, there is shown an example input pre-processor 6000 that is configured to process one or more response times 6010a-6010n for respective questions. For example, each value 6010a-6010n may be a respective time taken for a user to respond to a predetermined question. For example, the questions may be the questions asked during an input stage, as described above with respect to FIG. 5. Each response time 6010a is processed by a threshold logic 6012 configured to modify response times 6010 that are above a predetermined threshold value. For example, the threshold logic 6012 may remove response times 6010 that exceed the threshold value. Alternatively, the threshold logic 6012 may be configured to truncate response times that exceed the threshold value, for example by setting any response times that exceed the threshold value to the threshold value. The threshold value may be specific to a particular question or may apply to more than one question. The threshold value may be selected based upon previous response times, i.e. of other users. For example, the threshold value may be set based upon predetermined a quantile of all previously received response times for a particular question (or for all questions in aggregate). For example, the threshold may be the 90th percentile, or 95th percentile, of all response times for a particular question (or for all questions in aggregate). By processing the response times 6012 with threshold logic 6012, the input pre-processor 6000 can ensure that unusually long response times, which may indicate disengagement from the application, do not unduly influence the subsequent stages of the diagnostic.

Thresholded response times 6014a-6014n (collectively 6014) are output from the threshold logic 6012 and provided as input to response time processing logic 6016. The response time processing logic 6016 is configured to determine one or more outputs 6018a-6018n (collectively 6018) based upon the response times. For example, the response time processing logic 6016 may determine average (for example mean, mode or median) response times. For example, mean response times may be determined for all of the thresholded response times 6014 and/or separate mean response times may be determined for respective subsets of the response times. For example, where the response times 6010 relate to multiple distinct sets of questions (e.g. multiple questionnaires) mean response times may be determined for each of the sets of questions. The response time processing logic 6016 may further determine a variation in the thresholded response times 6014. Again, a variation in thresholded response times may be determined between all response times and/or between respective subsets of response times. It will be appreciated that other outputs may be determined based on the response times, such as median response times, modes of response times, maximum response times, shortest response times, etc. The outputs 6018 may be one or more of the outputs 3013.

The relative speed with which users answer different questions has been found to indicate their certainty in those answers, which may be useful in some diagnostic areas, such as medical diagnostics including mental health diagnostics. In order to reveal relative response speed for a given user between different questions, the response time processing logic 6016 may further calculate a z-score (i.e. standard score) for each response time 6010 of an individual user, using the individual's response times to calculate the population mean. This removes user-specific characteristics in response times and allows assessment of the relative response times between different questions for a user, which might reveal their certainty in specific answers. The outputs 6018 may therefore also include one or more z-scores of the response times 6010.

Other inputs 3010 may be received and may be processed in other ways by the input pre-processing stage 3012. For example, in addition to response times, other behavioural indicators may be received. For example, in addition to individual question response times, inputs 3010 may include times to first interaction with a particular subset of questions, total time to submission of an answer, numbers of changes of an answer, typing speed (e.g. measured in seconds per character) and number of deleted characters. Additionally, as described above, one or more of the inputs 3010 may include measurements made by the user or on the user, such as physiological characteristics of the user.

In some example implementations, one or more of the input pre-processors 3012a-3012n are configured to processed at least some of the received inputs 3010 using Principal Component Analysis (PCA) to identify a predetermined number of principal components (for example the top two or top ten principal components). For example, one of the input pre-processors 3012a-3012n may be configured to determine, using PCA a predetermined number of principal components of behavioural indicators and/or measurements received as input. The outputs 3012 may therefore include a number of principal components of a predetermined subset of the inputs. Principal Component Analysis may be performed using any widely available PCA algorithms/packages as will be readily apparent to the skilled person. Processing of the response times may occur at the user device 1010 or at the server 1030. Performing at least some processing of the response times at the user device 1010 may advantageously reduce the amount of data that is transmitted to the server 1030 over the network, reducing both bandwidth and increasing speed. In this way, further stages of the diagnostic may be implemented more quickly and efficiently.

In some examples, the inputs 3012 may include answers to a predetermined set of questions (“binary questions”) for which the permitted input is binary (e.g. yes/no). Example questions may relate to demographics, or to the nature of the problem to be diagnosed. The input pre-processors may generate a one-hot encoding to indicate the answers to the predetermined set of “binary questions”. In some examples, only some of the predetermined set of binary questions may be presented to the user, for example in dependence upon answers to other questions, or the result of processing carried out on others of the inputs 3010 (for example as described above). As such, the one-hot encoding may further indicate whether a particular question is asked. That is, each question may have two bits within the one-hot encoding, where one of the two bits encodes whether the question was asked, and the other of the two bits encodes the answer to the question. It will be appreciated that the answers may be formatted in other ways than a one-hot encoding vector. Encoding of answers to questions may be performed at the user device 1010 or at the server 1030. Encoding the answers to the questions at the user device may advantageously reduce the volume of data that is sent over the network, reducing both bandwidth and increasing speed. In this way, further stages of the diagnostic may be implemented more quickly and efficiently.

Referring again to FIG. 3a, it is to be understood that one or more stages of the system 3000 need not be included in all example implementations. For example, in one example implementation, the input pre-processing stage 3012 may be omitted and the inputs may be provided directly to the preliminary diagnostics model 3013.

One or more of the inputs 3010 (and in some implementations all of the inputs 3010) may be provided directly to the preliminary diagnostic model 3014. The preliminary diagnostic model 3014 is a classifier configured (i.e. trained) to determine a problem classification from the received inputs and to provide an output 3018 representing the problem classification. The preliminary diagnostics model 3014 may be a pre-trained machine learning classifier, trained on a training set of data relevant to the problem domain of the diagnostic. The preliminary diagnostics model 3014 may use any appropriate classification techniques. In one advantageous example, the preliminary diagnostics model 3014 may use a Gradient Boosting algorithm with decision trees as the weak learners, but may use any other appropriate classifier such as Bayesian classifiers.

The processing of the inputs 3010 performed by input pre-processing stage 3012 enables the inputs to be provided to the preliminary diagnostics model 3014 in tabular format. For example, while an input 3010 may be free text or audio, after processing by, e.g. the input pre-processor 4000, to output classes 4018, the classes can easily be represented in tabular format. The output of tabular format inputs 3013 enables a Gradient Boosting decision tree classifier to provide a particularly efficient implementation, thereby reducing both processing time and processing resources need to pre-process the inputs 3013, while providing an accurate estimates of the problem classifications 3018. The gradient boosting classifier algorithm may be, for example, an XGBoost algorithm. A regularised gradient boosting algorithm such as XGBoost may be particularly beneficial to enable parallelization of processing decisions within the decision tree, allowing for the question answers to be processed more quickly and with more efficient use of processing resources. In other examples, the classifier may use gradient boosting techniques such as CatBoost, LightGMB or others as will be known to those skilled in the art.

In an example in which the preliminary diagnostics model 3014 uses an XGBoost Gradient Boosting algorithm with decision trees as a weak learners, an example implementation may use the following hyperparameters:
Learning rate (eta): 0.01
Maximal depth of trees (max_depth): 14
Gamma: 22.4
Alpha: 1
Subsample ratio of columns by tree (colsample_bytree): 0.99
Subsample ratio of columns by level (colsample_bylevel): 0.88

The maximal depth of trees indicates the maximal depth of any decision tree, the learning rate indicates the step size shrinkage used in updates to prevent overfitting-after each boosting step, the weights of new features are obtained and the learning rate shrinks the feature weights to make the boosting process more conservative. Gamma indicates a minimum loss reduction required to make a further partition on a leaf node of the decision tree—larger gamma values cause the gradient boosting model more conservative. Alpha is an L1 regularization term on weights-increasing values of alpha will make model more conservative. Subsample ratio of columns by level indicates the subsample ratio of columns for each level of the tree. Subsample ratio of columns by tree is the subsample ratio of columns when constructing each tree.

The preliminary diagnostics model 3014 may be trained using any appropriate loss function. For example, where the preliminary diagnostics model 3014 uses a gradient boosting classifier, the loss function may be a multi-class log loss function. Training data for training the preliminary diagnostics model 3014 may comprise a dataset that includes values for the inputs 3010 and corresponding ground truth diagnosis labels assigned by experts. For example, a suitable training set may be obtained from historical data to obtain suitable inputs with expert-assigned diagnoses based on the facts corresponding to those inputs. It will be appreciated that the exact training set will depend upon the particular domain of the diagnostic that the application facilitates.

The preliminary diagnostic model 3014 may be multi-class classifier or a single class classifier such that the output 3018 may include a single classification or a list of possible classifications. The outputs 3018 may comprise an indication of a probability (or a confidence score or value) of the determined classification(s) and may further include a confidence score.

The output 3018 is provided to the output generator 3020 which is configured to generate an output 3022 comprising one or more further questions or sets of questions to present to the user. In particular, the application has access to a question database (or a plurality of question databases), for example stored in the storage 1040. The question database comprises a plurality of questions and/or sets questions each associated with a particular one or more of the possible problem classes identifiable by the preliminary diagnostic model 3014. Where the preliminary diagnostic model 3014 outputs a single classification, the output generator 3020 may determine whether there are any questions in the question database that are associated with the single classification and output those questions for presentation to the user.

Where the output 3018 comprises a list of possible classes together with a probability that the problem described by the inputs 3010 belong to that class, the output generator 3020 may select the most highly ranked (by probability) of the possible classes until the cumulative probability of the selected classes reaches a cumulative probability threshold. Put another way, the output generator 3020 may select the minimum number of possible classes having a cumulative probability that exceeds the cumulative probability threshold. It will be appreciated that the predetermined threshold may be selected based upon the requirements of the particular problem domain. By way of example only, the predetermined threshold may be 90%. By way of illustration only, if the output 3018 indicated:
class 1: 50%
class 2: 42%
class 3: 7%
class 4: 1%
class 4: 0%
   the output generator 3020 would select classes 1 to 2, having a cumulative probability of 92%. Similarly, where the output 3018 comprises a confidence score, the output generator 3020 may determine whether one or more questions or sets of questions are associated with one or more classes having a confidence above a confidence value threshold. For the selected classes, the output generator 3020 determines whether one or more questions or sets of questions from a question database that are associated with the selected classes. For example, each question or set of questions may be stored with an associated identifier identifying one or more problem classes. In the above example, the output generator 3020 may determine whether there are any questions or sets of questions that are associated with classes 1 and 2. The output generator 3020 generates an output 3022 comprising the determined one or more questions or sets of questions. The output generator 3020 may also determine whether the determined questions or sets of questions have already been presented to the user, for example during the input stage, as described above with reference to FIGS. 5 and 6. The output generator 3020 may remove questions or sets of questions which have already been presented to the user from the output 3022. In some examples, the output generator 3020 may also include other questions or sets of questions in the output 3022. For example, the output generator 3020 may determine whether any of the predicted classes have an individual probability that exceed an individual question threshold. Using the example above, if the second threshold had a value of 5%, the output generator 3020 may determine whether there are any questions or sets of questions in the question database that are associated with the class 3 and may include any such questions in the output 3022. Alternatively or additionally, the output generator 3020 may select a predetermined number of classes ranked immediately below the classes selected based upon the first threshold. For example, the output generator 3020 may be configured to select up to the next two most highly ranked classes. In the example above, the output generator 3020 may be configured to select classes 3 and 4.

Where there are multiple sets of questions associated with determined class(es), the output generator may perform a de-duplication operation to remove questions that are duplicated across multiple sets of questions. In this way, the diagnostic application may reduce the amount of data that needs to be sent between the user device 1010 and the server 1030 over the network while also improving the speed with which the diagnostic may be performed.

The questions indicated in the output 3022 may be presented to the user using the user interface, e.g. the chat interface depicted in FIG. 2. For example, the output 3022 may cause the application to retrieve the selected questions from the question database and to provide these to the user interface for presentation to the user, e.g. by transmitting the determined questions from the server 1030 to the user device 1010 over the network.

The output 3022 may include additional information. For example, the output 3022 may include some or all of the output 3018. For example, the output 3022 may include the classes selected by the output generator 3020 from the output 2018. The output 3022 may further include a cumulative probability and/or confidence score of the selected classes. It will be appreciated that the output 3022 may include any or all of the other outputs generated by the components of the system 3000, such as one or more outputs from the input stage 3012.

It will be appreciated that any or all of the pre-processing stage 3012 may be executed at one or more of the user devices 1010. As such, input data received at an input may be processed at the client device before being transmitted to the server 1030, either for further input pre-processing or for processing by the preliminary diagnosis model 3014.

Referring to FIG. 7, there is depicted an example of a diagnosis system 7000 that may be used in combination with the system 3000. The diagnosis system 7000 includes diagnosis logic 7020 configured to receive inputs 7010a-7010n (collectively 7010) and to determine, based on the inputs, a diagnosis of one or more problems characterised by the inputs. While a single diagnosis system 7000 is depicted in FIG. 7, multiple diagnosis systems may be provided. For example, diagnosis systems for respective possible problem classes.

The inputs 7010 may comprise user answer to the questions output by the system 3000, including answers received during the input stage and answers received in response to questions indicated in the output 3022. The inputs 7010 may further comprise some or all of the outputs 3022. The diagnosis logic 7020 may determine and output a diagnosis 7022 in accordance with any of a number of techniques. It will be appreciated that the particular techniques used may depend upon the particular problem domain. In one example, the diagnosis logic 7020 determines a positive diagnosis of a problem by scoring the answers to sets of questions relating to that problem. The diagnosis logic 7020 may then determine a positive diagnoses of that problem if the score meets or exceeds a threshold. Similarly, the diagnosis logic 7020 may determine a negative diagnosis of a problem if the score is below the threshold.

To provide another example, the diagnosis logic 7020 may comprise one or more machine learning models configured to process the inputs 7010 and to output a diagnosis. For example, the diagnosis logic 7020 may comprise one or more machine learned classifiers configured to determine a diagnosis of one or more problems from the inputs 7010. The machine learned classifiers may be implemented similarly to either of the machine learned classifiers described above in connection with FIGS. 3, 4 and 5. A machine learned classifier for use in the diagnosis logic 7020 may be trained on historical data that includes the values for inputs 7010 together with associated diagnoses provided by experts. By providing diagnosis logic 7020 that includes a machine learned classifier, the diagnosis logic 7020 may determine diagnoses of problems based on relationships between answers to questions that may not be taken into account when scoring individual sets of questions.

Responsive to generating the output diagnosis 7022, the output 7022 may be transmitted to the user device 1010 of the user. Additionally or alternatively, the output diagnosis 7022 may be transmitted to a third party. For example, depending on the problem domain, the output may be transmitted to an engineer, a clinician, a medic, an emergency service or a manager of the user. In some example implementations, the application may be configured to determine, based on the output 7022 whether to transmit the output 7022 to a third party and/or to determine to which of a plurality of third parties the output will be transmitted.

The output 7022 may be provided as an input to action logic 7024. The action logic 7024 be configured to select an action to perform responsive to the output 7022 and/or to cause performance of an action in response to the output 7022. For example, as described above, the action may be transmission of the output 7022 to a user or a third party. Where the application is configured to diagnose a problem with a device or machine, the output logic 7024 may be configured to automatically cause a visit to be scheduled by a repair engineer. Additionally or alternatively, the action logic 7024 may be configured to select, generate and/or output maintenance instructions to assist the user or third party to perform corrective action on the device or machine. For example, the action logic 7024 may be configured to query one or more maintenance databases to determine an appropriate course of action response to the output diagnosis.

Where the application is configured to diagnose a physical or mental health condition, the action logic 7024 may be configured to generate a treatment plan, schedule appointments with clinicians or to establish a communication with an emergency service. For example, the action logic 7024 may be configured to establish, in response to a diagnosis that indicates urgent care is required, a communication channel between the user and an emergency service or between a clinician and an emergency service and may transmit the output 7022 to the emergency service. For example, in response to confirming or disconfirming a diagnosed condition, a user may be allocated to a predetermined treatment pathway depending on any diagnosis or diagnoses confirmed base on the output 7022. For example, allocation to a treatment pathway may be performed by the action logic 7024. A predetermined treatment pathway is the route to which is patient is seen by a mental health care professional. There may be several different pre-programmed treatment pathways. For example a treatment pathway for patients that are prioritised for early treatment so that they are seen by a mental health care professional within 2 weeks, or a treatment pathway for patients whose condition is relatively mild and who could be seen by a mental health care professional within a longer wait time of 8 weeks. The mental health care service may be informed of the user and their allocated treatment pathway by the action logic 7024. The user can then be seen by a mental health care professional according to their allocated treatment pathway. The action logic 7024 may be configured to prioritize some users for treatment based on the output 7022.

As described above, one example use for the techniques described herein is in the diagnosis of medical conditions, such as mental illness. Mental illness is currently the largest cause of disability in the United Kingdom, where approximately one in four adults experience a mental health problem each year at a cost of around £105 billion. The number of referrals for psychological therapy for common mental health disorders has increased significantly over the last 8 years however service capacities have not increased at the same rate. This supply-demand imbalance can create long waiting times for patients, which is known to negatively impact health outcomes. In the UK, there is a wait time of around 20 days for an initial assessment and diagnosis from a mental health care professional. There is then an average wait time of 10 weeks before the first treatment session in the UK. Some patients need treatment more quickly than others in view of the particular nature and severity of their mental health conditions. However, the current system is inadequate in prioritising these patients for treatment and ensuring that they are treated quickly. The presence of a mental health condition may be defined by presentation of a number of symptoms which may only be determined by the patient answering specific questions or sets of questions (questionnaires). A number of clinical questionnaires have been developed that are considered to be the gold standard in diagnosis of mental health conditions. A current approach used is for a therapist to complete an initial clinical assessment via a phone call (typically lasting around one hour). However, during diagnosis patients are often presented with multiple clinical questionnaires that are not necessarily related to their particular condition. The questionnaires can be lengthy and, in view of the number and length of the questionnaires, they can be off-putting to fill in, which leads to some patients not completing them, and the relevant data not being available for analysis. The supply-demand imbalance for treatment by mental health care professionals is not unique to the UK and is prevalent in other countries. There is therefore a desire to improve the route to patients receiving treatment from a mental health care professional.

In overview, having the machine learning model predict the initial diagnoses, which are then confirmed or disconfirmed by the computer-implemented method, provides a better allocation of medical resources since human therapists are not required to initially diagnose and triage the patients, freeing up time for human therapists to provide therapeutic treatment instead. Furthermore, the techniques described herein allow the highest priority users to be identified and prioritised for treatment, e.g. via a treatment pathway. Furthermore, selecting one or more initial diagnoses for further assessment allows the further assessment to be tailored to the specific diagnoses of the user. Furthermore, using machine learning to predict a plurality of initial diagnoses of the user, and then selecting one or more of these initial diagnoses for the further assessment, provides a particularly accurate way of confirming the correct diagnosis or diagnoses for the user. When the confirmed diagnosis or diagnoses comprise a predetermined condition, then the user may be automatically prioritised for earlier treatment by a mental health care professional via the predetermined treatment pathway over other users who do not have the confirmed diagnosis or diagnoses comprising the predetermined condition. The predetermined condition may comprise a diagnosis of a predetermined mental and/or behavioural disorder such as depression, generalised anxiety disorder, obsessive-compulsive disorder (OCD), post-traumatic stress disorder (PTSD), social phobia, health anxiety, panic disorder and specific phobias. Selecting one or more of the plurality of initial diagnoses for the further assessment may comprise selecting one, two, three, four or more than four of the initial diagnoses of the plurality of the initial diagnoses for the further assessment. Selecting more than one diagnosis for the further assessment increases the accuracy of the method resulting in a correct diagnosis compared to selecting just one diagnosis for the further assessment.

The step of using the at least one machine learning model to predict the plurality of initial diagnoses of the user may comprise using a third machine learning model to predict a second set of preliminary diagnoses of a mental and/or behavioural disorder for the user and a second set of preliminary confidence values for the first set of preliminary diagnoses, wherein the second set of preliminary confidence values comprise a confidence value of a preliminary diagnosis being correct for each of the preliminary diagnoses of the second set of preliminary diagnoses, and inputting the second set of preliminary confidence values into the second machine learning model for predicting the plurality of initial diagnoses. The structured approach of having a second machine learning model which takes as input the output from a first and optionally a third machine learning model increases the accuracy in making the predictions of the initial diagnoses since the predictions for the diagnoses are refined by the successive models. One or each of the first machine learning model, the second machine learning model and the third machine learning model may comprise a gradient boosting decision tree. The first machine learning model may operate on user data from a first data modality, and the third machine learning model may operate on user data from a second data modality. Using different data modalities captures different reflections of the user's mental health and this diversity increases the accuracy in making the predictions of the initial diagnoses. Using the second machine learning model to predict the plurality of initial diagnoses, and optionally the confidence values, may comprise the second machine learning model operating on user data from a third data modality. The user data may comprise sensor data of the user. Optionally, the sensor data includes digital biomarkers such as response speed, typing speed, number of deletions in text. As different mental health disorders are associated with different underlying cognitive characteristics (e.g. apathy in depressed patients versus hyper alertness in patients with anxiety disorders), inclusion of digital biomarkers enables the machine learning models to determine mental health disorders based upon those characteristics. The additional information may comprise the user's answers to questions from clinically recognised mental health questionnaires such as the PHQ-9 and/or the GAD-7 questionnaire. Advantageously, combining machine learning models to form an initial hypothesis (i.e. equivalent to clinical judgement) and then administering known clinically validated questionnaires to confirm this hypothesis meets the gold standard for assessing mental health diagnoses, while enabling this decision to be made in real time and through this reduce the overall wait time for patients to receive mental health treatment and thus improve their overall care. The known clinically recognised mental health questionnaires have been validated for decades and enable the quantification of the severity of specific mental health problems that the patient experiences. The step of performing the further assessment may further comprise collecting additional information from the user in relation to the selected two or more initial diagnoses, and optionally wherein collecting additional information comprises issuing the user with questions that are specific to the selected two or more initial diagnoses, wherein the questions are from one or more clinically recognised mental health questionnaires such as the PHQ-9 and/or the GAD-7 questionnaire. The computer-implemented method may further comprise a step of deduplication in which the questions are reviewed prior to being issued to the user to remove any questions that may otherwise have been issued to the user two or more times. The plurality of initial diagnoses comprise any or all of the following initial diagnoses: depression; generalised anxiety disorder, mixed anxiety-depressive disorder; social phobia; obsessive compulsive disorder (OCD); post-traumatic stress disorder (PTSD); panic disorder; health anxiety; specific phobias; agoraphobia; eating disorder; other disorder. It will be appreciated that these initial diagnoses are only examples of initial diagnoses of mental and/or behavioural disorders, and the mental and/or behavioural disorders, and the total number thereof, may vary.

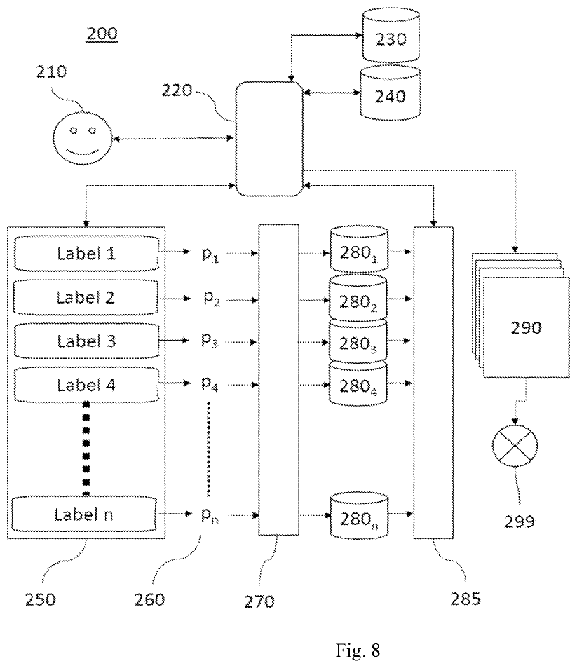

Referring now to FIG. 8, there is shown an overview of a system 200 that provides a dialogue system for interacting with a user through a user interface (such as the chat interface 100). It is to be understood that features described above in connection with FIGS. 3 to 7 may be used in the system 200.

A user 210 that uses the interface shown in FIG. 2 interacts with a dialogue system engine 220 (which may also be referred to as a chatbot engine) that provides a user interface (e.g. the interface shown in FIG. 2) via a user interface (e.g. a touch screen, microphone and/or speaker) of a user device 1010 operated by the user 210. As described above, other or multiple mechanisms for collecting data or input from a user 210 may be employed, allowing for information or queries to be presented to the user 210 and responses collected from the user 210.

The dialogue system engine 220 is provided in communication with a medical database 230 and a question database 240. The medical database 230 and question database 240 may be stored in storage 1040. The medical database 230 can comprise one or many databases of information which include information about the user 210 that can be retrieved by the dialogue system engine 220 as and when required. For example the medical database 230 can be a database held by a doctor's surgery, or a hospital, or government health records (or multiple of these). In some implementations, no medical database 230 is provided.

The medical database 230 can be used to obtain relevant information about the user 210. Obtaining information about the user 210 may require the user 210 to be sufficiently authenticated by the dialogue system engine 220 and/or the medical database 230. This method of obtaining of relevant information can prevent the user 210 having to manually enter or confirm information that has already been collected and stored in the medical database 230.

The question database 240 contains one or more questions or sets of questions and prompts that can be displayed to a user. These questions and prompts can optionally be pre-determined and structured in such a way to follow one or more sequences of questions, or to ask a set of generic questions to collect a base set of information from a user or a set of free text responses from a user. For example, a base set of information can be collected from a user following a series of prompts in order to authenticate and obtain information on the user from the medical database 230, following which free text is collected from the user in response to one or a series of questions (which depending on the implementation may be structured, or generic, or based on the retrieved information from the medical database 230).

In this example, the dialogue system engine 220 is also in communication with a trained model 250, deduplicated output 285 from the trained model 250 and also a set of diagnosis models 290.

The information collected from the dialogue system engine 220 and medical database 230 is then provided to the trained model 250. Using this input information, the trained model 250 makes a set of predictions for a set of labels. In this example, each label is a mental and/or behavioural disorder, (in the Figure, labels 1 to n) and outputs a probability for each label and/or confidence score or value for each probability 260. A threshold check 270 is performed on the set of probabilities and confidence scores 260 and only labels with probability and/or confidence scores above a predetermined threshold are output as triggers from the threshold check process 270. The threshold check process 270 may be implemented by the output generator 3020 and may alternatively or additionally operate as described in connection with FIG. 3a above. For example, the threshold check process 270 may be configured to output those labels with a cumulative probability and/or confidence score above a threshold, for example 90%. The triggers output by the threshold check process 270 trigger question database 2801-280, to be queried to output a set of questions for each label 280, for example by the output generator 3020. The question databases 2801-280n may be specific question databases for each label, or may be a single question database such as the question database 240. The output sets of questions may be deduplicated 285 to remove duplicate questions (for example, where multiple databases 280 are triggered to output questions to be presented to the user 210 and/or multiple sets of questions contain duplicate questions). The deduplicated questions are then output to the dialogue system engine 220 by the deduplication process 285 to be presented in turn to the user 210 and responses collected. Where de-duplication is not performed, the output sets of questions may be provided directly to the dialogue system engine 220.

The trained model 250 is implemented in this example using a machine learning model such as a gradient boosted decision tree or a probabilistic Bayesian deep learning model providing a probability metric, alongside each prediction of an initial diagnosis, i.e. for each label/problem descriptor/disorder. This has the advantage of increased interpretability of the predictions made and allowing the use of thresholding 270 of the confidence level of each of the set of the predictions output 260 by the model 250. For example, the trained model 250 may be the trained model 3014. While not depicted in FIG. 8, as described above in connection with FIG. 3a, the inputs to the trained model 250 may first be processed by an input pre-processing stage 3012. Alternative methods of selecting and presenting questions to users are possible. For example, a chat interface may be used and both a first trained model 250 is used and one or a set of diagnosis models 290 are used, but alternative mechanisms to use the output probability and/or confidence values from the first trained model 250 can be used in order to select sets of disorder specific questions to present to a user via a chat interface, and alternative mechanisms to provide the answers (and optionally questions) to the one or set of diagnosis models 290 can be used.

In some example implementations, the sets of disorder specific questions comprise medically validated symptom questionnaires such as the Patient Health Questionnaire-9 (PHQ-9) and/or the Generalised Anxiety Disorder Assessment (GAD-7). In other examples, the sets of disorder specific questions may be replaced or supplemented with other tasks for the user to complete. For example, the first trained model 250 may be trained to output questions with free text answers. Alternatively or additionally, one or more cognitive tasks may be selected for the user to perform in order to obtain one or more digital biomarkers. Alternatively or additionally, further actions may be selected in order to obtain speech samples, video samples, physiological measures, etc.

In the example of FIG. 8, following receipt of the responses to the questions output by the deduplication process 285 presented to the user 210, the responses (and optionally all other previously gathered information and/or the questions presented to the user) are provided to the one or more diagnosis models 290. For example, the diagnosis models 290 may be implemented as described above in connection with the diagnosis logic 7020. Each diagnosis model 290 may be trained to make a diagnosis pertaining to a particular mental health characteristic or condition. Each model of the diagnosis models 290 makes a diagnosis and these diagnoses are output 299. In alternative implementations where the diagnosis models 290 comprise a single combined model or plural combined models, rather than a set of models where each model makes a separate diagnosis per condition, these one or more combined models will output one or more diagnoses 299. As described in connection with FIG. 7, a diagnosis may instead be based only on a scoring of the responses to the questions output by the deduplication process 285.

In the example of FIG. 8, the specific questions databases 280 and each model of the diagnosis models 290 are disorder specific. The trained model 250 is a machine learned model that predicts one or more problem descriptors (i.e. one or more labels), a problem descriptor being also referred to herein as an initial or preliminary diagnosis, and then the delivery of the disorder-specific questions 280 to the user 210 is used to confirm one or more diagnoses 299 using the diagnosis models 290. The output of the trained model 250 is used to justify the automated administering of one or more disorder-specific questionnaires 280, e.g., to confirm the initial hypothesis about possible disorders.

One or more of the diagnosis models 290 can optionally be hand-crafted, for example to calculate scores and/or sum scores provided as answers to the questions, for example as described above in connection with FIG. 7.

Training data is collected to train the machine learning models 250 to output a probability of the user 210 presenting one of a set of mental health problems from data input into the dialogue system engine 220 by the user 210 (and optionally, using information extracted from a medical database 230). Where the Improving Access to Psychological Therapies “IAPT” problem descriptor ICD-10 codes (with 8 classes) is used, historical patient records can be used to pre-train the weights on the machine learning models to predict the probability distribution over at least the most common IAPT problem descriptor codes. In some implementations, further data can be collected as the models are used and further training can be performed, for example including patient records and clinical outcomes data and any digital biomarkers generated using any or any combination of natural language processing, sentiment analysis and/or analysis of typing patterns (as these data streams can be used to predict symptoms of mental illness) and/or interactions with the app (e.g. response times). Optionally instead or as well, other information can be used such as information collected passively by the user's computing device including sensor data such as accelerometery, video, audio, temperature, light intensity, GPS, touch screen events, cursor movements, haptic feedback, electrocardiogramography.

Training data is collected to train the diagnosis models 290 to make/predict a diagnosis for a set of disorders, each model of the diagnosis models 290 being disorder specific.

The output 299 includes the information provided by the user 210 via the dialogue system engine 220 and optionally any relevant information extracted from the medical database 230, which can then be reviewed against the output diagnosis from the diagnosis models 290 by a medical practitioner.

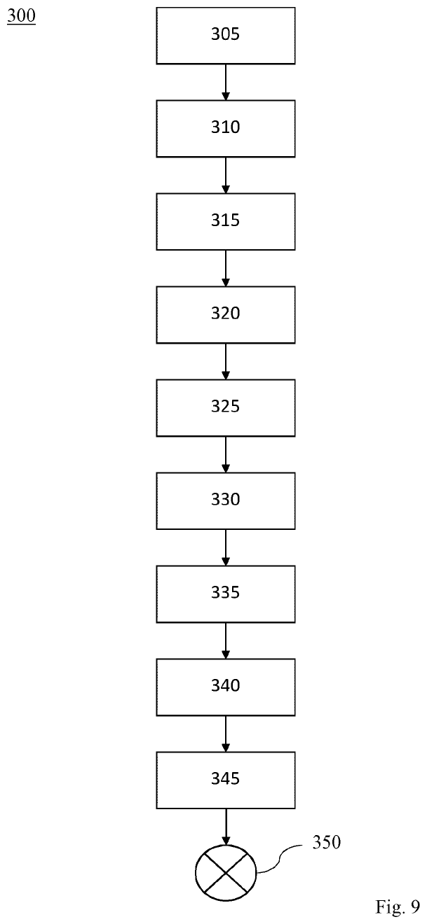

Referring now to FIG. 9, an example diagnosis process 300 will now be described in more detail below.

In the example process 300, the initial step is for the user to login to the system at step 305. This involves authenticating their identity sufficiently in order to allow access to medical database information and store personal and medical data about them securely. In example implementations, logging in can be done via a login screen, or using authentication on the user device, or via the chat interface using any appropriate authentication systems. In some implementations, step 305 may be omitted.

Once the user is logged in and authorised (if necessary), if access to one or more medical databases containing historic and current medical data about the user is provided, information can be retrieved about the user. Questions to be presented to the user can then be retrieved at step 310, either one by one or in batches, for presentation to the user via a user interface. Other interfaces can alternatively be used. In some implementations, the questions to be presented to the user may be stored within the dialogue system engine.

Next, the questions are presented to the user at step 315 via the chat interface. Responses to the questions can be determined by the question type, for example some questions might be multiple-choice questions while others may require free text input. Additionally or alternatively, user interface manipulation may be used for user data input, such as moving a virtual slider along a sliding scale or interacting with a virtual game (such as tapping/clicking on animated virtual targets on a screen). Responses are collected via the dialogue system interface to each question presented. The structure of the questions may follow a decision tree approach, where one answer to a multiple choice question may prompt one set of follow up questions while another answer will prompt a second set of follow up questions (where the first and second sets of questions may contain some common questions or completely different questions, and in some instances an answer to a multiple choice question might prompt multiple of the sets of questions to be presented to the user).

The dialogue system interface may instead be implemented using natural language processing to determine the input and/or information (sometimes termed the intent) required to complete one or more tasks, jobs or functions. More specifically, to determine the intent, the dialogue system interface is configured to ask questions of the user and analyse the (free text) responses to identify the information the dialogue system wants to obtain in order to determine the intent, i.e. to complete a task, job or function such as, for example, to answer a question or provide a numerical rating. A variety of different pieces of information might be required to determine the intent, or for example if there are multiple tasks, jobs or functions being performed. In the conversation with the user, each different piece of information and/or intent is determined via the dialogue system interface using natural language processing of the (free text) responses from the user via the sequence of questions presented to the user (if and as required) until the process is complete and the multiple tasks, jobs and/or functions are completed. For example, the Amazon® LEX customer service dialogue system framework (see https://aws.amazon.com/lex/features which is hereby incorporated by reference) can be used to provide the dialogue system interface.

The questions and responses (along with any optional medical data obtained from the one or more medical databases) may then be provided to the trained model to make a set of predictions 320 with confidence values for each prediction, each prediction for a specific disorder or condition.

Using the set of predictions with confidence values, and applying a pre-determined threshold to select only predictions having a sufficient level of confidence, one or more sets of disorder-specific questions are selected 325 for presentation to the user via the dialogue system interface.

In the example process 300, a step 330 of de-duplicating the questions is performed where multiple sets of questions have been selected in step 325. This step is optional and may not be present in all implementations. Then a step 335 is performed to present the disorder specific questions to the user and obtain responses and answers. Again, the responses depend on the question type as per step 315. Then a step 340 of providing the responses and answers, and the questions presented, for both steps 315 and 335, to a trained set of models to make a diagnosis using a model per disorder is performed. Alternatively, one or more combined models can be used (optionally alongside the per-disorder model(s)) where the combined models output diagnoses for more than one disorder, the combined models being jointly-trained across training data for more than one disorder.

The multiple diagnoses per disorder are then output at a step 350. The diagnoses may be output for review by a medical practitioner. Further automated steps can be performed such as to retrieve pertinent information and present this to the user; and/or to present options for treatment to the user for the user to select and use/book; and/or to automatically book appointments or treatments. In these other implementations, optionally these further automated steps can be performed without a medical practitioner reviewing the output 350.

Table 1 provides an example of diagnoses which may be predicted by a machine learning model or models for a user, including initial diagnoses output by machine learning models such as the preliminary diagnosis model 3014 or the trained model 250 and diagnoses output by diagnosis logic 7020 and models 290.

TABLE 1

| Disorder | Probability |
|---|---|
| Depression | 0.50 |
| Social Phobia | 0.25 |
| Post-traumatic stress disorder | 0.02 |
| Panic disorder | 0.15 |
| Generalised anxiety disorder | 0.02 |
| Mixed anxiety-depressive disorder | 0.01 |
| Obsessive compulsive disorder (OCD) | 0.00 |
| Health Anxiety | 0.05 |
| Specific phobias | 0.00 |
| Agoraphobia | 0.00 |
| Eating disorder | 0.00 |
| Other disorders | 0.00 |

Other disorders is a generalised category for disorders different than those specifically listed in the other initial diagnoses. It will be appreciated that the initial diagnoses shown in Table. 1 are only examples of mental and/or behavioural disorders that can be predicted by the machine learning models, and the mental and/or behavioural disorders, and the total number thereof, may vary.

The machine learning model or models may also provide a confidence value for each of these initial diagnoses. For the example user represented in Table. 1, the user has an initial diagnosis of depression with a probability of 0.5. This means that there is a relatively high probability that the user has depression. The user also has an initial diagnosis of post-traumatic stress disorder with a confidence value of around 0.02. The user has a lower probability of having post-traumatic stress disorder than depression.

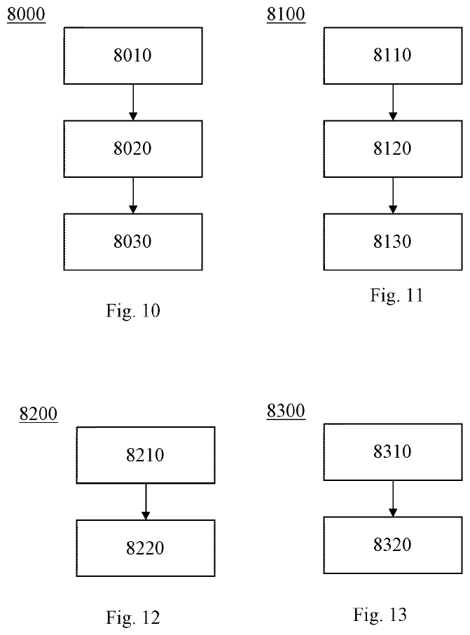

With reference to FIGS. 10 to 13, there is now described a number of example computer-implemented methods that may be performed by the systems described herein.

Referring to FIG. 10, there is shown a flowchart of a computer-implemented method 8000 that may be performed to process text or audio data received from a user. The method 8000 may be performed by an input pre-processor of an input pre-processor stage 3012. For example, the method 8000 may be performed by the input pre-processor 4000.

At a step 8010, the computer system implementing the method 8000 (for example one or more of the server 1030 and the user device 1010) receives from the user an input comprising text or audio data. The input may be received from the user device over a network. The text or audio data may be free-text or free-audio. That is there may not be any constraints on the informational content of the text or audio that the user provides. It will be appreciated that there may be constraints on the length of the text or audio input while still be considered to be free of informational constraints. In an example in which the method 8000 is used in a diagnosis of a mental health condition, the input may be provided in response to a question provided in a user interface (such as the chat interface of FIG. 2). In an example in which the method 8000 is used in a diagnosis of a mental health condition, the input may be received in response to a prompt such as “What is the main problem that brought you here today? Be sure to include specific feelings, behaviours or thoughts that are bothering you.”

At step 8020, the one or more inputs may be provided to one or more models configured to reduce the dimensionality of the input received at step 8010. The one or more models to reduce the dimensionality of the input may comprise an embedding model, such as the embedding model 4012. Additionally, or alternatively, the one or more models may comprise one or more classifiers, such as the classifiers 4016a, 4016b. The processing at step 8020 outputs data representing the input received at step 8010 but with reduced-dimensionality. For example, the output may be an embedding, or may advantageously be one or more classes. Each class may have a confidence value associated with it. In an example in which the method 8000 is used in a diagnosis of a mental health condition, the classes may be one or more of the classes shown in Table 1.

The output of step 8020 is provided, at a step 8030, to a preliminary diagnosis model configured (e.g. trained) to predict and output diagnoses and associated confidence values for the user inputs received at step 8010. For example, the preliminary diagnosis model used at step 8020 may be the preliminary diagnosis model 3014. In a particularly advantageous example, the output of step 8020 is one or more classes which may be represented in tabular form and the preliminary diagnosis model is a gradient boosting decision tree. In an example in which the method 8000 is used in a diagnosis of a mental health condition, the preliminary diagnoses output at step 8030 may be those shown in Table 1.

Referring to FIG. 11, there is shown a flowchart of a computer-implemented method 8100 that may be performed to process question responses received from a user. The method 8100 may be performed by an input pre-processor of an input pre-processor stage 3012. For example, the method 8100 may be performed by the input pre-processor 5000.

At a step 8110, the computer system implementing the method 8100 (for example one or more of the server 1030 and the user device 1010) receives from the user an input comprising a user's answer to specific questions or sets of questions that have been presented using a user interface. In an example in which the method 8100 is used in a diagnosis of a mental health condition, the specific questions may be one or more clinically validated questionnaires on which diagnoses of mental health conditions is dependent. For example, the questions or sets of questions may be one or more questions or sets of questions that are mandated by clinical guidelines to be administered to a patient. For example, the response received at step 8110 may be responses to the following questionnaires:
PHQ-9 (Kroenke et al., 2001): measuring symptoms of depression
GAD-7 (Spitzer et al., 2006): measuring symptoms of generalised anxiety
IAPT Phobia scales: measuring different phobia related symptoms
WSAS (Mundt et al., 2002): measuring general functional impairment

At step 8120, the input received at step 8110 is pre-processed to reduce the dimensionality of the input. For example, the input may be processed at step 8120 by the input pre-processor 5000. In a particularly advantageous example, the inputs received at step 8110 may be represented in tabular format, and the input pre-processor applied at step 8120 may use a gradient boosting decision tree to classify the inputs and output one or more classes. Each class may have a confidence value associated with it. In an example in which the method 8000 is used in a diagnosis of a mental health condition, the classes may be one or more of the classes shown in Table 1.

Referring to FIG. 12, there is shown a flowchart of a computer-implemented method 8200 that may be performed to process sensor data received from a user or user device (such as the user device 1010). The method 8200 may be performed by an input pre-processor of an input pre-processor stage 3012. For example, the method 8200 may be performed by the input pre-processor 6000.

At a step 8210, the computer system implementing the method 8200 (for example one or more of the server 1030 and the user device 1010) receives an input comprising sensor data. In an example in which the method 8100 is used in a diagnosis of a mental health condition, the sensor data may include digital biomarkers which are collected via digital devices such as a user's mobile phone or a wearable device. The sensor data may include any or all of the following:
Reaction time: the time it takes to respond to a binary or non-binary question. Reaction time can be used to make inferences about the cognitive processes happening in the user.
Typing speed: Typing speed can indicate vigour or exerted effort, and these dynamics can be useful for identifying mood/depressive symptoms.
Delete button presses: Deleting text might indicate uncertainty, indecisiveness and self doubt.

At step 8220, the input is provided to feature extraction logic configured to extract and output features from the sensor data. For example, the feature extraction logic may include the threshold logic 6012 and the response time processing logic 6016, and/or other logic for processing sensor data as described in connection with FIGS. 1 to 7 above. In an example in which the method 8200 is used in a diagnosis of a mental health condition reaction times may be processed in a handcrafted way using measures of interest such as mean response times and variation in response time in order to capture general characteristics of the patient. The handcrafted summary statistics of the response times may be augmented by principal components analysis over all sensor data to derive patterns in the behavioural variables. For example, the first 10 principal components may be used as a summary measure of general characteristics of a patient's cognitive characteristics. In addition, response times are indicators for certainty of a decision, whereby patients respond faster when they are more certain in their answer. Thus, the relative speed with which patients answer different questions provides an indication of their certainty in those answers. To reveal relative response speed for a given patient between different questions, PHQ-9, GAD-7, WSAS and Phobia scale response times may be z-scored within each patient to removes patient specific characteristics in response times and allow provide relative response times between different questions for a patient.

Referring to FIG. 13, there is shown a flowchart for processing other input data. At step 8310, the computer system implementing the method 8300 (for example one or more of the server 1030 and the user device 1010) receives an input comprising answers to a predetermined set of questions (“binary questions”) for which the permitted input is binary (e.g. yes/no) or otherwise constrained. In an example in which the method 8300 is used in a diagnosis of a mental health condition, example questions and possible answers may include:
Age: integer
Gender-Answers: “Male (including transmale)”, “Female (including transfemale)”, “Non-binary”
Ethnicity-Answers: “White”, “Non-white”
Disability status-Answers: “Disability present”, “No Disability”
Long-term medical condition-Answers: “Long Term medical condition present”, “No long term medical condition”
Use of alcohol to regulate mood-Answers: “Yes”, “No”
Use of substance to regulate mood-Answers: “Yes”, “No”
Receiving mental health treatment from other institution: “Yes”, “No”

At step 8320, the input received at step 8310 is provided to a pre-processor configured to generate and output an encoding to indicate the answers to the predetermined set of questions. For example, a vector may be output with a value for each question. Alternatively, a one-hot encoding may be generated for those questions having a binary answer. As described above, the encoding generated at step 8320 may further indicate whether a particular question is asked.

By providing one or more machine learning models for respective inputs, the different machine learning models can be specific to the type of data that they operate on. As such, the input pre-processing methods described herein enable the inputs to be assessed in the most appropriate way for the type of data of the input, therefore improving the efficiency of determining diagnoses based on the inputs and improving the accuracy of those diagnoses. Further, as described above, the input pre-processing methods described herein may reduce the amount of data that needs to be transmitted between devices.

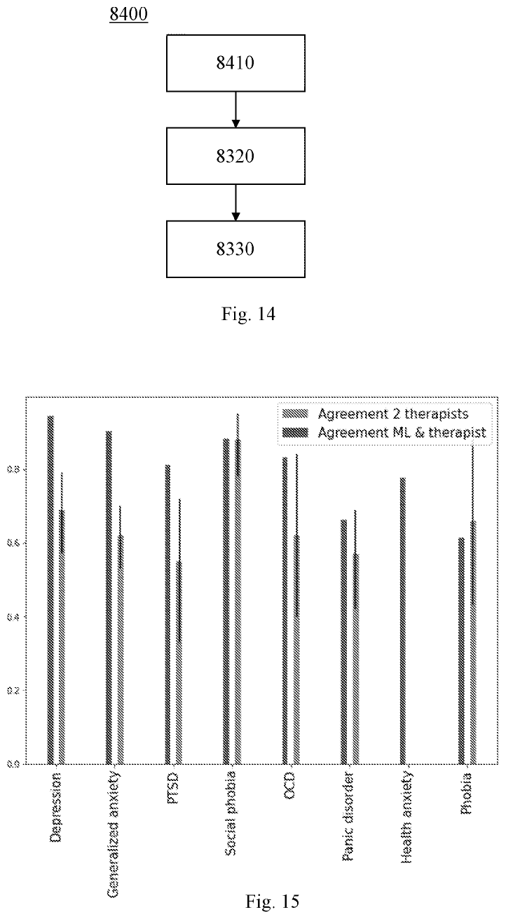

Referring to FIG. 14, there is shown a flowchart for an example process 8400 for processing outputs from one or more of the processes 8000-8300. The method 8400 may be performed by the preliminary diagnostics model 3014 and the output generator 3020.

At step 8410, the computer system implementing the method 8500 (for example one or more of the server 1030 and the user device 1010) receives an input comprising the outputs from one or more of the processes 8000-8300 described above. At step 8410, the inputs are processed to determine a preliminary (or initial) diagnosis. For example, the processing at step 8410 may be performed by the preliminary diagnosis model 3014 or the trained model 250 as described above. The processing performed at step 8420 may use a gradient boosting decision tree to classify the inputs received at step 8410 and output one or more classes. Each class may have a probability associated with it. In an example in which the method 8400 is used in a diagnosis of a mental health condition, the classes may be one or more of the classes shown in Table 1.

The preliminary diagnosis generated at step 8320 is passed to a step 8330 and the preliminary diagnosis is processed to determine and output one or more questions or sets of questions to output to a user. For example, the processing at step 8330 may be performed by the output generator 3020 and/or threshold operation 285. The selection may involve selecting the two initial diagnoses which have the highest confidence values. For the example of Table. 1, this would involve selecting the initial diagnoses of depression and social phobia. As described above, alternative selection methods are possible, for example instead of selecting the two initial diagnoses with the highest confidence values, the computer-method may comprise selecting the three or four or more initial diagnoses which have the highest confidence values. Alternatively, the computer-implemented method may comprise selecting the initial diagnoses which have a confidence value greater than a predetermined threshold such as 50%. For the example of Table. 1, this would involve selecting the initial diagnoses of depression for the further assessment. Alternatively, the computer-implemented method may comprise selecting the initial diagnoses with the highest confidence values until a cumulative confidence value exceeds a threshold, such as 90%. In the example of Table 1, this would involve selecting the initial diagnoses of Depression, Social Phobia and Panic Disorder.

For illustration purposes, in an example in which the method 8400 is used in a process to diagnose a mental health condition, the processing of step 8330 may select from the following medically validated questionnaires.
Depression: Patient Health Questionnaire-9 (PHQ-9)
Generalised anxiety: Generalised Anxiety Disorder-7 (GAD-7)
Social phobia: Social Phobia Inventory (SPIN)
Panic disorder: Panic Disorder Severity Scale (PDSS)
OCD: Obsessive-Compulsive Inventory revised (OCI-R)
PTSD: PTSD Checklist for DSM-5 (PCL-5)
Health anxiety: Health Anxiety Inventor (HAI-18)
Specific Phobia: Severity Measure for Specific Phobia (SMSP)

The processing of step 8330 may perform other steps prior to outputting the list of questions, as described above with reference to the questions selector 3020. For example, the processing of step 8330 may include removing questions which have already been presented to a user, removing duplicate questions, and selecting only questions or sets of questions corresponding to a preliminary diagnosis class that has a sufficiently high probability (and/or confidence) or a group having a cumulative probability greater than a threshold.

The processing at step 8330 may output any or all of considered diagnoses including their determined probabilities, the list of diagnoses having a cumulative probability greater than a threshold, the cumulative probability that the correct diagnosis was included in the list of the considered diagnoses (i.e. the cumulative probability of the classes that were selected) and a list of selected questions or sets of questions.

Furthermore by providing a structure of success machine learning models as described above enables successive models to refine the diagnoses, thereby improving the accuracy in determining the preliminary diagnoses on which the questions or sets of questions are selected. Furthermore, processing a plurality of different data modalities that each capture different representations of the problem to be diagnosed increases the accuracy of the preliminary diagnoses on which the question selection is based.

The machine learning models described herein are trained by supervised learning. Where the machine learning models are used in a diagnostic method for diagnosing a mental health condition, the training data includes data from users who are known to have a diagnosis or diagnoses which has been confirmed by a mental health care professional and data corresponding to the inputs described above.

An exemplary system, together with performance data is now discussed. It will be appreciated that any techniques described in connection with the exemplary model may be used more generally. In one exemplary implementation, a system was developed, trained and tested on a data set of 18,278 patients which had been collected through use of a diagnosis of the type described herein in IAPT services of the UK's National Health Service (NHS). Once the model was trained, the system was tested on an additional set of 2,557 patients which were newly collected in a prospective study. Finally, the model performance was compared against the reliability of human clinical diagnoses. The exemplary system comprised a free-text input pre-processing module (e.g. such as the input pre-processing module 4000), a standardised questionnaire pre-processing module (e.g. such as the input pre-processing module 5000), a specific question pre-processing module, and a behavioural indicators pre-processing module (e.g. such as the input pre-processing module 6000).

Input to the free-text input pre-processing module comprised answers to the question: “What is the main problem that brought you here today? Be sure to include specific feelings, behaviours or thoughts that are bothering you.” Moreover, patients were asked whether they take prescribed medication and if yes, the name of the medication was asked for. Since the form of medication could be an indicator for presence of specific issues, this was also provided as an input to the preliminary diagnosis model.

The free-text input pre-processing module comprised a BERT embedding model (“bert-base-nli-mean-tokens”) processed the free-text input (on the level of the whole paragraph the user has inputted) to generate an embedding output. The output of the embedding model was provided as input to a pre-trained classifier comprising a feedforward neural network with two hidden layers and the activation of the output layer of the pre-trained classifier (the class probability of each category before parsing through a softmax function) was saved for output to the preliminary diagnosis model. The first model was trained and tested on a total of 591,812 data points from mental health forums (80% training set, 10% validation set for early stopping of the training and 10% test set), covering the following topics/categories which were mutually exclusive: Depression, Generalised anxiety disorder, Social anxiety, OCD, PTSD, Health anxiety, Panic disorder, Phobia, Agoraphobia, Eating disorder, Addiction, Bi-polar disorder, Self-harm & suicide, Borderline personality disorder, Alcoholism. In order to account for an imbalance of observations for different classes, the less common categories were oversampled in the training set in order to match the number of cases in the most common category. The pre-trained classifier was trained to predict the topic/category for the mental health forum.

On a test set of the mental health forum data, the model achieved the following performance (F1-score):
Depression: 0.431
Generalised anxiety disorder: 0.223
Social anxiety: 0.65
OCD: 0.585
PTSD: 0.585
Health anxiety: 0.717
Panic disorder: 0.473
Phobia: 0.269
Agoraphobia: 0.585
Eating disorder: 0.81
Addiction: 0.676
Bi-polar disorder: 0.469
Self-harm & suicide: 0.626
Alcoholism: 0.797
Borderline personality disorder: 0.386

Indicating that this model was able to predict the topic of a mental health forum post based on the text of this post.

The free text input pre-processor comprised a second pre-trained classifier. The second pre-trained classifier was trained and tested on 453,000 data points from mental health forums (80% training set, 10% validation set for early stopping of the training and 10% test set), covering the following topics/categories: Depression, Generalised anxiety disorder, Social anxiety, OCD, PTSD, Health anxiety, Panic disorder, Phobia, Agoraphobia, Eating disorder, Addiction, Bi-polar disorder. In order to account for the imbalance of observations for different classes, less common categories in the training set were oversampled in order to match the number of cases in the most common category. The second pre-trained classifier comprised one hidden layer trained to predict the topic category for the mental health forum.

An n-gram bag of word embedding (using 3-grams) was generated, with a dictionary defined by the most common words in each category which were not included in the most common words of all categories (e.g. the 500 most common words in each specific category which were not in the 2000 most common words of all categories) in order to select for specific words that uniquely indicate a certain mental health diagnosis. Finally, the transformer (eg. BERT) based embedding and the bag-of-words based embedding were concatenated for each free-text input, resulting in a 7781 dimensional representation for each text input.

The concatenated embedding was provided to the second pre-trained classifier network.

On a test set of the mental health forum data, the model achieved the following performance (F1-score):
Depression: 0.854
Generalised anxiety disorder: 0.3244
Social anxiety: 0.798
OCD: 0.798
PTSD: 0.771
Health anxiety: 0.853
Panic disorder: 0.602
Phobia: 0.306
Agoraphobia: 0.619
Eating disorder: 0.8
Addiction: 0.872
Bi-polar disorder: 0.504

Indicating that this model was able to predict the topic of a mental health forum post based on the text of a post from the forum.

The standard questionnaire pre-processing module was trained on historic patient data of IAPT patients (a total of 32,894 IAPT patients), including their item level answers to the PHQ-9, the GAD-7, the IAPT phobia scale as well as their total score of the WSAS as well as their mental health diagnosis. A gradient boosting model (XGBoost) was provided to predict the diagnosis based on answers to the items of the standardised questionnaires. The diagnosis was categorised into the following categories: Depression, Generalised anxiety disorder, Health anxiety, Panic disorder, Social anxiety, OCD, PTSD, Mixed anxiety and depressive disorder, Eating disorder, Addiction (alcohol and other substances), Agoraphobia, Other: any other mental health diagnosis. The XGboost algorithm had the following hyperparameters: Maximal depth=10, Maximum of estimator trees: 5000, Learning rate: 0.01, Gamma: 5, Alpha: 1, Colsample_bylevel: 0.5. The historic patients data set was split into a training set (80% of the data), a validation set for early stopping (10% of the training set) and a test set (10%). In order to account for an imbalance of observations for different classes, the less common categories were oversampled in the training set in order to match the number of cases in the most common category. On the test set of the historic patient data, the model achieved the following performance (F1-score):
Depression: 0.63
Generalised anxiety disorder: 0.49
Health anxiety: 0.03
Panic disorder: 0.03
Social anxiety: 0.3
OCD: 0.07
PTSD: 0.18
Phobia: 0.29
Mixed anxiety and depressive disorder: 0.01
Eating disorder: 0.0
Addiction (alcohol and other substances): 0.0
Agoraphobia: 0.0.
Other: any other mental health diagnosis: 0.24

The behavioural indicators pre-processor was configured to define, for every response time, a 95% quantile of the collected data was established. Any response time above this value was set to this value, as extremely long response times might indicate a disengagement from the dialogue system and could influence the model output too strongly. In addition to constraining the maximum response time, the behavioural indicators were preprocessed in two ways. First, reaction times were processed to determine a plurality of features of interest including mean response times and variation in response time to capture some general characteristics of the patient. In order to augment the handcrafted summary statistics of the response times, a principal components analysis was also performed over all behavioural metrics, taking the first 10 principal components as a summary measure of general characteristics of a patient's cognitive characteristics. The relative speed with which patients answer different questions indicates their certainty in those answers. In order to reveal relative response speed for a given patient between different questions, the PHQ-9, GAD-7, WSAS and Phobia scale response times were z-scored within each patient to assess the relative response times between different questions for a patient which might reveal their certainty in specific answers.

The specific questions pre-processor was configured to generate a one-hot encoding that included whether the question had been asked to the patient.

To train the preliminary diagnosis model, a dataset with input features and corresponding labels (i.e. diagnoses assigned by clinicians) was used. Overall, a dataset of 18,278 patients that had been assigned a diagnosis was available for training and testing the ML-model. As diagnosis, the ICD-10 code for each patient was provided by the IAPT services. The preliminary diagnosis model was trained to predict one of the following categories (named here with their respective ICD-10 code):
Depression: This was a combined category including the International Classification of Diseases, Revision 10 (ICD-10) codes of “Depressive episode—F32” as well as “Recurrent Depressive disorder—F33”
Generalized anxiety disorder—F41.1
Social phobias—F40.1
Post-traumatic stress disorder—F43.1
Obsessive-compulsive disorder—F42
Panic disorder [episodic paroxysmal anxiety] without agoraphobia—F41.0
Health anxiety:
   Hypochondriacal disorder, unspecified—F45.2
Phobia: Specific (isolated) phobias—F40.2
   Agoraphobia—F40.0
Eating disorders—F50
Mixed anxiety and Depressive disorder—F41.2
Other:
   Any other Diagnosis
      Thus, the model was trained to distinguish between 12 different potential diagnoses categories.

A gradient boosting algorithm was utilised. This model was set up as a multi-class classification model with a “multi:softprob” objective function. The following model parameters were chosen
Learning rate (eta): 0.01
Maximal depth of trees (max_depth): 14
Gamma (gamma): 22.4
Alpha (alpha): 1
Subsample ratio of columns by tree (colsample_bytree): 0.99
Subsample ratio of columns by level (colsample_bylevel): 0.88

Given an imbalance in the occurrence of different diagnoses (depression represents 49.5% of all diagnoses while specific phobia only represents 0.6% of all diagnoses), it was not straightforward to optimising for overall accuracy and also achieve high performance for the less common diagnoses (which would not affect the overall unweighted average of accuracy much). In order to achieve both a strong overall performance while also achieving high performance for every single diagnosis category, the objective function was defined as a mix between overall accuracy and high performance for less common diagnoses. Accuracy is defined as the percentage of times in which the actual diagnosis (i.e. from an expert) was within the list of diagnoses checked by the preliminary diagnosis model. The objective function was defined as the combination of the micro averaged accuracy score (i.e. the overall accuracy independent of the diagnosis) and macro averaged accuracy score (i.e. the accuracy for each individual diagnosis category whereby all of these were averaged with equal weight, meaning that diagnoses with many and with few counts contributed equally to this average).

The following is the selected ranges for each hyperparameters that was used in the search process:
Max_depth: [7, 15] (integers)
Gamma: [0, 40] (real number)
Colsample_bytree: [0.5, 1] (real number)
Colsample_bylevel: [0.5, 1] (real number)

The best selected hyperparameters resulting from this search process were:
Max_depth: 14
Gamma: 22.4
Colsample_bytree: 0.99
Colsample_bylevel: 0.88
   This is the setting of hyperparameters which was used in the exemplary system. The model was trained and tested using a 10-fold, stratified cross validation. The algorithm was trained based on a multi-class log loss function. The training data for each fold of the cross validation was further split into a training set (90% of data) and a validation set (10% of data) which was used for determining an early stopping criterion (i.e. at early stop if the model prediction on the validation set had not improved within the last 10 steps) to avoid overfitting to the training set. Within the training set (but not the validation or test set) the less common diagnoses were oversampled (a sampling with replacement from the existing data points was applied) in order to match the count of cases in the most common diagnosis (i.e. depression). This was conducted to ensure that that algorithm would not over optimise for the most common mental health diagnoses and neglect less common diagnoses when optimising.

On the 10-fold cross validation, the model achieved an overall accuracy of 93.5% (CI=[93.1%, 93.9%]) in identifying the correct diagnosis for the 8 most common mental health disorders (depression, generalised anxiety disorder, social phobia, PTSD, OCD, panic disorder, health anxiety, specific phobia). Notably, both the average performance of the algorithm, and also the performance of the algorithm for each of these diagnoses was determined, to ensure the system is able to correctly check for each of these most common diagnoses.

TABLE 2
Accuracy of ML-model for detecting the eight most common mental
health diagnoses in IAPT in 10-fold cross validation

| | Number of | ML-model mean over folds, |
|---|---|---|
| | diagnoses in | with max and min accuracy |
| Diagnosis | dataset | over folds |
| Depression | 8966 | mean = 95.9%; (min: 94.4%, |
| | | max = 97.2% over folds) |
| Generalised | 4974 | mean = 96.8% (min: 95.7%, |
| anxiety disorder | | max = 98.4% over folds) |
| Social phobia | 872 | mean = 88.0% (min: 83.9%, |
| | | max = 91.9% over folds) |
| PTSD | 834 | mean = 83.0% (min: 77.3%, |
| | | max = 86.7% over folds) |
| OCD | 416 | mean = 73.1% (min: 63.4%, |
| | | max = 82.9% over folds) |
| Panic disorder | 338 | mean = 74.0% (min: 58.8%, |
| | | max = 88.2% over folds) |
| Health anxiety | 308 | mean = 76.0% (min: 64.5%, |
| | | max = 90.3% over folds) |
| Specific phobia | 109 | mean = 46.8% (min: 27.2%, |
| | | max = 72.7% over folds) |

In a prospective evaluation, to ensure that the model was not overfitted to the training and test data, test data for 2,557 new patients was processed. The model performed to the same accuracy as in the test and training data set when run in this prospective evaluation, achieving an overall accuracy of 94.2% (CI=[93.3%, 95.1%]) for detecting the 8 most common mental health problems. Similarly to the test and training dataset, this accuracy did hold for each of the relevant diagnoses.

TABLE 3
Accuracy of ML-model for detecting the eight most common
mental health diagnoses in IAPT in prospective study

| | Number of | |
|---|---|---|
| | diagnoses in | Correctly identified |
| Diagnosis | dataset | diagnoses by ML-model |
| Depression | 1,432 | 1,396 |
| | | (97.5%) |
| Generalised anxiety disorder | 672 | 643 |
| | | (95.7%) |
| Social phobia | 145 | 133 |
| | | (91.7%) |
| PTSD | 132 | 110 |
| | | (83.3%) |
| OCD | 61 | 50 |
| | | (82.0%) |
| Panic disorder | 46 | 31 |
| | | (67.4%) |
| Health anxiety | 53 | 41 |
| | | (77.4%) |
| Specific phobia | 16 | 8 |
| | | (50%) |

FIG. 15 is a chart showing a comparison of the exemplary system to human performance. The first bar in each category (x-axis) indicates agreement between the preliminary diagnosis output by the preliminary diagnosis model and diagnoses assigned by therapists during treatment. The second bar in each category indicates the reliability between independent therapists based on data presented in two studies (Reed et al. 2018, Tolin et al. 2018). The error bars represent the confidence intervals reported in these studies.

Machine learning is the field of study where a computer or computers learn to perform classes of tasks using the feedback generated from the experience or data gathered that the machine learning process acquires during computer performance of those tasks.

Typically, machine learning can be broadly classed as using either supervised or unsupervised approaches, although there are particular approaches such as reinforcement learning and semi-supervised learning which have special rules, techniques and/or approaches.

Supervised machine learning is concerned with a computer learning one or more rules or functions to map between example inputs and desired outputs as predetermined by an operator or programmer, usually where a data set containing the inputs is labelled.

Unsupervised learning is concerned with determining a structure for input data, for example when performing pattern recognition, and typically uses unlabelled data sets.

Reinforcement learning is concerned with enabling a computer or computers to interact with a dynamic environment, for example when playing a game or driving a vehicle.

Various hybrids of these categories are possible, such as “semi-supervised” machine learning where a training data set has only been partially labelled. For unsupervised machine learning, there is a range of possible applications such as, for example, the application of computer vision techniques to image processing or video enhancement.

Unsupervised machine learning is typically applied to solve problems where an unknown data structure might be present in the data. As the data is unlabelled, the machine learning process is required to operate to identify implicit relationships between the data for example by deriving a clustering metric based on internally derived information. For example, an unsupervised learning technique can be used to reduce the dimensionality of a data set and attempt to identify and model relationships between clusters in the data set, and can for example generate measures of cluster membership or identify hubs or nodes in or between clusters (for example using a technique referred to as weighted correlation network analysis, which can be applied to high-dimensional data sets, or using k-means clustering to cluster data by a measure of the Euclidean distance between each datum).

Semi-supervised learning is typically applied to solve problems where there is a partially labelled data set, for example where only a subset of the data is labelled. Semi-supervised machine learning makes use of externally provided labels and objective functions as well as any implicit data relationships. When initially configuring a machine learning system, particularly when using a supervised machine learning approach, the machine learning algorithm can be provided with some training data or a set of training examples, in which each example is typically a pair of an input signal/vector and a desired output value, label (or classification) or signal. The machine learning algorithm analyses the training data and produces a generalised function that can be used with unseen data sets to produce desired output values or signals for the unseen input vectors/signals. The user needs to decide what type of data is to be used as the training data, and to prepare a representative real-world set of data. The user must however take care to ensure that the training data contains enough information to accurately predict desired output values without providing too many features (which can result in too many dimensions being considered by the machine learning process during training and could also mean that the machine learning process does not converge to good solutions for all or specific examples). The user must also determine the desired structure of the learned or generalised function, for example whether to use support vector machines or decision trees.

The use of unsupervised or semi-supervised machine learning approaches are sometimes used when labelled data is not readily available, or where the system generates new labelled data from unknown data given some initial seed labels.

Machine learning may be performed through the use of one or more of: a non-linear hierarchical algorithm; neural network; convolutional neural network; recurrent neural network; long short-term memory network; multi-dimensional convolutional network; a memory network; fully convolutional network or a gated recurrent network allows a flexible approach when generating the predicted block of visual data. The use of an algorithm with a memory unit such as a long short-term memory network (LSTM), a memory network or a gated recurrent network can keep the state of the predicted blocks from motion compensation processes performed on the same original input frame. The use of these networks can improve computational efficiency and also improve temporal consistency in the motion compensation process across a number of frames, as the algorithm maintains some sort of state or memory of the changes in motion. This can additionally result in a reduction of error rates.

Developing a machine learning system typically consists of two stages: (1) training and (2) production.

During the training the parameters of the machine learning model are iteratively changed to optimise a particular learning objective, known as the objective function or the loss.

Once the model is trained, it can be used in production, where the model takes in an input and produces an output using the trained parameters.

During the training stage of neural networks, verified inputs are provided, and hence it is possible to compare the neural network's calculated output to then the correct the network is need be. An error term or loss function for each node in neural network can be established, and the weights adjusted, so that future outputs are closer to an expected result. Backpropagation techniques can also be used in the training schedule for the or each neural network.

The model can be trained using backpropagation and forward pass through the network. The loss function is an objective that can be minimised, it is a measurement between the target value and the model's output.

The cross-entropy loss may be used. The cross-entropy loss is defined as

$${L}_{CE} = - {\sum }_{c = 1}^{C} y * log ( s )$$

where C is the number of classes, y∈{0,1} is the binary indicator for class c, and s is the score for class c.

In the multitask learning setting, the loss will consist of multiple parts. A loss term for each task.

$$L ( x ) = {λ}_{1} {L}_{1} + {λ}_{2} {L}_{2}$$

where L₁, L₂ are the loss terms for two different tasks and λ₁, λ₂ are weighting terms.

Any system feature as described herein may also be provided as a method feature, and vice versa. As used herein, means plus function features may be expressed alternatively in terms of their corresponding structure.

Any feature in one aspect may be applied to other aspects, in any appropriate combination. In particular, method aspects may be applied to system aspects, and vice versa. Furthermore, any, some and/or all features in one aspect can be applied to any, some and/or all features in any other aspect, in any appropriate combination.

It should also be appreciated that particular combinations of the various features described and defined in any aspects can be implemented and/or supplied and/or used independently.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively, or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “processor”, “computer” or “computing device” generally refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, logic, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a mark-up language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, the subject matter described in this specification can be implemented on a computer having a display device, e.g., a CRT (cathode ray tube) or LCD (liquid crystal display) monitor, for displaying information to the user and a keyboard and a pointing device, e.g., a mouse or a track-ball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework or other.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

A computing system can include clients and servers as illustrated in FIG. 1. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Below is a list of numbered statements relating to the invention:

1. A dialogue system comprising:
an input for receiving input data relating to a speech or text input signal originating from a user device;
an output for outputting audio or text information; and
one or more processors configured to:
   receive first input data at the input, the first input data indicating at least one problem;
   process the first input data at a first input pre-processing module comprising a first input pre-processing machine learning model configured generate a representation of the first input data and to generate a first input pre-processing module output based at least in part on the representation of the first input data;
   determine a preliminary diagnosis output comprising at least one preliminary diagnosis of the problem, comprising:
      processing the first input pre-processing module output at a preliminary diagnosis machine learning model configured to determine the preliminary diagnosis output based at least in part on the first input pre-processing module output;
   determine, based at least in part on the preliminary diagnosis output, at least one dialogue system output; and
   output, by way of the output of the dialogue system, the at least one dialogue system output.
      2. The dialogue system of statement 1, wherein the one or more processors are configured to:
receive second input data at the input, the second input data comprising a plurality of answers responsive to predetermined questions output by the dialogue system;
processing the second input data at a second input pre-processing module comprising a second input pre-processing machine learning model configured to generate a second input pre-processing module output, the second input pre-processing module output comprising a prediction of at least one problem based at least in part upon the second input pre-processing module output; and
wherein determining the preliminary diagnosis output comprises processing the second input pre-processing module output at the preliminary diagnosis machine learning model and the preliminary diagnosis machine learning model is configured to determine the preliminary diagnosis output based at least in part on the second input pre-processing module output.
   3. The dialogue system of statement 1, further comprising one or more sensors for receiving sensor input data measuring a characteristic of a user, wherein the one or more processors are configured to:
receive third input data received at the one or more sensors, the third input data comprising a plurality of sensor signals measuring a characteristic of the user;
process the third input data at a third input pre-processing module configured to generate a third input pre-processing module output comprising one or more principal components of the third input data;
wherein determining the preliminary diagnosis output comprises processing the third input pre-processing module output at the preliminary diagnosis machine learning model and the preliminary diagnosis machine learning model is configured to determine the preliminary diagnosis output based at least in part on the third input pre-processing module output.
   4. The dialogue system of statement 1, further comprising one or more sensors for receiving sensor input data measuring a characteristic of a user, wherein the one or more processors are configured to:
receive fourth input data received at the one or more sensors, the fourth input data comprising a plurality of sensor signals measuring a response time of a user when answering each of a plurality of questions output by the dialogue system;
processing the fourth input data at a fourth input pre-processing module configured to generate a fourth input pre-processing module output comprising at least one of an average response time, variation between one or more response times, a minimum response time and a maximum response time;
wherein determining the preliminary diagnosis output comprises processing the fourth input pre-processing module output at the preliminary diagnosis machine learning model and the preliminary diagnosis machine learning model is configured to determine the preliminary diagnosis output based at least in part on the fourth input pre-processing module output.
   5. The dialogue system of statement 1, wherein the one or more processors are configured to:
receive fifth input data comprising one or more answers to one or more questions represented by the at least one dialogue system output;
determine, based at least in part on the fifth input data, one or more further diagnoses of the problem; and
outputting, by way of the output, the one or more further diagnoses.
   6. The dialogue system of statement 5, wherein determining one or more further diagnoses of the problem comprises providing the fifth input data to a machine learning classifier trained to determine the one or more further diagnoses of the problem based upon the fifth input data.
   7. The dialogue system of statement 5, wherein the one or more processors are configured to:
cause, responsive to the one or more further diagnoses, an action to be taken or scheduled.
   8. The dialogue system of statement 7, wherein the one or more processors are configured to determine, responsive to the one or more further diagnoses, a priority and wherein the action is determined responsive to the priority.
   9. The dialogue system of statement 5, wherein the one or more processors are configured to establish, responsive to the one or more further diagnoses, a communication channel with a third party.
   10. The dialogue system of statement 1, wherein the preliminary diagnosis machine learning model comprises a gradient boosting decision tree classifier.
   11. The dialogue system of statement 1, wherein the preliminary diagnosis model was trained using an objective function defined by a combination of a micro averaged accuracy score and a macro averaged accuracy score.
   12. The dialogue system of statement 1, wherein the first input pre-processing module comprises a plurality of first input pre-processing machine learning models each configured to generate a respective representation of the first input data having a lower dimensionality than the first input data and each trained on a different dataset; and
the at least one processor is configured to generate the first input pre-processing module output based at least in part on the plurality of representations of the first input data.
   13. The dialogue system of statement 1, wherein the first input pre-processing module comprises at least one embedding machine learning model configured to generate an embedding of the first input and to provide the embedding as an input to the first input pre-processing machine learning model.
   14. The dialogue system of statement 1, wherein the first input pre-processing module comprises a classifier machine learning model configured to determine, based on the first input data, one or more categories of problem indicated in the first input data.
   15. The dialogue system of statement 1, wherein the preliminary diagnosis model is configured to determine a respective probability value for each of a plurality of categories; and
wherein the one or more processors are configured to:
   determine one or more of the plurality of categories based on the respective probability values; and
   determine the at least one dialogue system output by determining at least one dialogue system output associated with each of the determined one or more of the plurality of categories.
      16. The dialogue system of statement 15, wherein the one or more processors are configured to determine one or more of the plurality of categories by selecting a predetermined number of the plurality of categories having highest probability values.
      17. The dialogue system of statement 1, wherein
at least one of the one or more processors are part of a client device and at least one of the one or more processors are part of a server device;
at least a part of the first input pre-processing module is operated on the client device; and
the preliminary diagnosis model is operated on the server device.
   18. The dialogue system of statement 1, wherein the one or more processors are configured to:
receive a plurality of user inputs each having a different data modality;
provide each user input to a respective input pre-processing module configured to generate an output for inputting to the preliminary diagnosis model; and
wherein determining the preliminary diagnosis output comprises:
   processing each of the respective input pre-processing module outputs at the preliminary diagnosis machine learning model to provide the preliminary diagnosis output based at least in part on each of the respective input pre-processing module outputs.
      19. The dialogue system of statement 1, wherein the input data relates to mental health, the preliminary diagnosis output comprises a diagnosis of one or more mental health conditions and the one or more dialogue system outputs comprise questions for confirming or disconfirming the diagnosis of one or more mental health conditions.
      20. A method of generating output for a dialogue system, the method comprising:
receiving, at an input, input data relating to a speech or text input signal originating from a user device, the first input data indicating at least one problem;
processing, at one or more processors executing a first input pre-processing module comprising a first input pre-processing machine learning model, the first input data to generate a representation of the first input data and to generate a first input pre-processing module output based at least in part on the representation of the first input data;
determining, at the one or more processors, a preliminary diagnosis output comprising at least one preliminary diagnosis of the problem, the determining comprising processing, using a preliminary diagnosis machine learning model, the first input pre-processing module output;
determining, at the one or more processors and based at least in part on the preliminary diagnosis output, at least one dialogue system output; and
outputting, by way of an output, the dialogue system output.
   21. One or more non-transitory computer readable media, storing computer readable instruction configured to cause one or more computing systems to:
processing, at one or more processors executing a first input pre-processing module comprising a first input pre-processing machine learning model, first input data to generate a representation of the first input data and to generate a first input pre-processing module output based at least in part on the representation of the first input data, the first input data relating to a speech or text input signal originating from a user device, the first input data indicating at least one problem;
determining, at the one or more processors, a preliminary diagnosis output comprising at least one preliminary diagnosis of the problem, the determining comprising processing, using a preliminary diagnosis machine learning model, the first input pre-processing module output;
determining, at the one or more processors and based at least in part on the preliminary diagnosis output, at least one dialogue system output; and
outputting, by way of an output, the dialogue system output.
   22. A computer-implemented triage method, comprising:
receiving input data from a user;
using at least one probabilistic Bayesian deep learning models to predict a plurality of probabilities using the received input data, each probability associated with one of a plurality of problem descriptors and each probability comprising a confidence value;
selecting one or more sets of queries, each of the one or more sets of queries associated with each of the plurality of problem descriptors, wherein each set of queries is selected when the associated predicted probability of the problem descriptor exceeds a predetermined threshold;
requesting a set of responses from the user to the selected one or more sets of queries;
receiving the set of responses to the one or more selected sets of queries from the user; and
generating at least one diagnosis associated with at least one of the plurality of problem descriptors using the input data, the plurality of probabilities and the set of responses.
   23. The method of statement 22 wherein:
the input data received from the user comprises any or any combination of: selected answers from a plurality of predetermined answers; free text.
   24. The method of statement 22 or 23 wherein:
the input data is augmented to further comprise relevant information about the user extracted from at least one medical database, optionally wherein the input data is used to identify relevant information about the user in the at least one medical database.
   25. The method of statement 22, 23 or 24 wherein:
the input data and set of responses is received from the user via a chat interface.
   26 The method of any of statements 22 to 25 further comprising:
deduplicating the selected one or more sets of queries.
   27. The method of any preceding of statements 22 to 26 wherein:
the step of selecting one or more sets of queries, each of the one or more sets of queries associated with each of the plurality of problem descriptors, wherein each set of queries is selected when the associated predicted probability of the problem descriptor exceeds a predetermined threshold;
comprises selecting one or more sets of queries, each of the one or more sets of queries associated with each of the plurality of problem descriptors, wherein each set of queries is selected when the associated predicted probability and confidence value of the problem descriptor exceeds a predetermined threshold.
   28. The method of any of statements 22 to 27 further comprising:
outputting the generated at least one diagnosis.
   29. A system for performing computer-implemented triage, operable to:
receive input data from a user;
use at least one probabilistic Bayesian deep learning models to predict a plurality of probabilities using the received input data, each probability associated with one of a plurality of problem descriptors and each probability comprising a confidence value;
select one or more sets of queries, each of the one or more sets of queries associated with each of the plurality of problem descriptors, wherein each set of queries is selected when the associated predicted probability of the problem descriptor exceeds a predetermined threshold;
request a set of responses from the user to the selected one or more sets of queries;
receive the set of responses to the one or more selected sets of queries from the user, and
generate at least one diagnosis associated with at least one of the plurality of problem descriptors using the input data, the plurality of probabilities and the set of responses.

1. A computer-implemented method for automated diagnostics, the method comprising:
receiving, at an input of a diagnostics system, input data relating to a speech or text input signal originating from a user device, the first input data indicating at least one problem;
processing, at one or more processors, the first input data using a first input pre-processing module comprising a first input pre-processing machine learning model, to generate a representation of the first input data and to generate a first input pre-processing module output based at least in part on the representation of the first input data;
processing, at the one or more processors, the first input pre-processing module output using a preliminary diagnosis machine learning model to determine a preliminary diagnosis output comprising at least one preliminary diagnosis of the problem;
determining, at the one or more processors and based at least in part on the preliminary diagnosis output, at least one dialogue system output;
outputting, by way of an output of the diagnostics system, the dialogue system output;
receiving, at the input of the diagnostics system, additional input data responsive to the dialogue system output;
processing, at the one or more processors, the additional input data to determine one or more further diagnoses; and
outputting, by the output of the diagnostics system, an indication of the one or more further diagnoses.

2. The method claim 1, further comprising:
receiving second input data at the input, the second input data comprising a plurality of answers responsive to predetermined questions output by the diagnostics system;
processing the second input data at a second input pre-processing module comprising a second input pre-processing machine learning model to generate a second input pre-processing module output, the second input pre-processing module output comprising a prediction of at least one problem based at least in part upon the second input pre-processing module output; and
wherein determining the preliminary diagnosis output comprises processing the second input pre-processing module output at the preliminary diagnosis machine learning model and the preliminary diagnosis output is based at least in part on the second input pre-processing module output.

3. The method of claim 1, further comprising:
receiving third input data from one or more sensors, the third input data comprising a plurality of sensor signals measuring a characteristic of a user;
processing the third input data at a third input pre-processing module configured to generate a third input pre-processing module output comprising one or more principal components of the third input data;
wherein determining the preliminary diagnosis output comprises processing the third input pre-processing module output at the preliminary diagnosis machine learning model and the preliminary diagnosis machine learning model is configured to determine the preliminary diagnosis output based at least in part on the third input pre-processing module output.

4. The method of claim 1, further comprising:
receiving fourth input data from one or more sensors, the fourth input data comprising a plurality of sensor signals measuring a response time of a user when answering each of a plurality of questions output by the dialogue system;
processing the fourth input data at a fourth input pre-processing module configured to generate a fourth input pre-processing module output comprising at least one of: an average response time, variation between one or more response times, a minimum response time and a maximum response time;
wherein determining the preliminary diagnosis output comprises processing the fourth input pre-processing module output at the preliminary diagnosis machine learning model and the preliminary diagnosis machine learning model is configured to determine the preliminary diagnosis output based at least in part on the fourth input pre-processing module output.

5. The method of claim 1, wherein determining one or more further diagnoses of the problem comprises providing the fifth input data to a machine learning classifier trained to determine the one or more further diagnoses of the problem based upon the fifth input data.

6. The method of claim 1, further comprising:
causing, responsive to the one or more further diagnoses, an action to be taken or scheduled.

7. The method of claim 6, further comprising determining, responsive to the one or more further diagnoses, a priority; and
wherein the action is determined responsive to the priority.

8. The method of claim 6, wherein the action comprises at least one of:
allocating a user of the user device to a treatment pathway for treatment by a clinician;
scheduling an appointment with a clinician;
establishing a communication channel with an emergency service; and
generate and/or output one or more instructions and/or treatment plan actions for the user.

9. The method of claim 1, wherein the preliminary diagnosis machine learning model comprises a gradient boosting decision tree classifier.

10. The method of claim 1, wherein the preliminary diagnosis model was trained using a multi-class objective function defined by a combination of a micro averaged accuracy score and a macro averaged accuracy score, wherein the micro averaged accuracy score was defined by an overall accuracy diagnoses output by the preliminary diagnosis model independent of an accuracy of individual diagnosis categories and the macro averaged accuracy score were defined by accuracies of individual diagnosis categories output by the preliminary diagnosis model and averaged with equal weight.

11. The method of claim 1, wherein the first input pre-processing module comprises a plurality of first input pre-processing machine learning models each configured to generate a respective representation of the first input data having a lower dimensionality than the first input data and each trained on a different dataset; and
the method comprises generating the first input pre-processing module output based at least in part on the plurality of representations of the first input data.

12. The method of claim 1, wherein the first input pre-processing module comprises at least one embedding machine learning model configured to generate an embedding of the first input and to provide the embedding as an input to the first input pre-processing machine learning model.

13. The method of claim 1, wherein the first input pre-processing module comprises a classifier machine learning model configured to determine, based on the first input data, one or more categories of problem indicated in the first input data.

14. The method of claim 1, wherein the preliminary diagnosis model is configured to determine a respective probability value for each of a plurality of categories, each respective probability value indicating a confidence that category is associated with the input data; and
wherein the method further comprises:
determining one or more of the plurality of categories based on the respective probability values; and
determining the at least one dialogue system output by determining at least one dialogue system output associated with each of the determined one or more of the plurality of categories.

15. (canceled)

16. (canceled)

17. The method of claim 1, wherein at least a part of the first input pre-processing module is operated on a client device; and
the preliminary diagnosis model is operated on a server device.

18. The method of claim 1, wherein
the input data is one of a plurality of user inputs each having a different data modality;
the method comprises:
providing respective ones of the plurality of user inputs to respective input pre-processing modules, each input pre-processing module configured to generate a respective input pre-processing module output for inputting to the preliminary diagnosis model; and
wherein determining the preliminary diagnosis output comprises:
processing each of the respective input pre-processing module outputs at the preliminary diagnosis machine learning model to provide the preliminary diagnosis output based at least in part on each of the respective input pre-processing module outputs.

19. The method of claim 1, wherein the input data relates to mental health, the preliminary diagnosis output comprises at least one diagnosis of one or more mental health conditions and the one or more dialogue system outputs comprise questions for confirming or disconfirming the at least one diagnosis of one or more mental health conditions.

20. The method of claim 1, wherein determining at least one dialogue system output, further comprises:
selecting one or more sets of questions relating to the at least one preliminary diagnosis.

21. (canceled)

22. One or more computer readable media, storing computer readable instructions configured to cause one or more processors to perform a method of automatic diagnosis, the method comprising:
receiving, at an input of a diagnostics system, input data relating to a speech or text input signal originating from a user device, the first input data indicating at least one problem;
processing, at one or more processors, the first input data using a first input pre-processing module comprising a first input pre-processing machine learning model, to generate a representation of the first input data and to generate a first input pre-processing module output based at least in part on the representation of the first input data;
processing, at the one or more processors, the first input pre-processing module output using a preliminary diagnosis machine learning model to determine a preliminary diagnosis output comprising at least one preliminary diagnosis of the problem;
determining, at the one or more processors and based at least in part on the preliminary diagnosis output, at least one dialogue system output;
outputting, by way of an output of the diagnostics system, the dialogue system output;
receiving, at the input of the diagnostics system, additional input data responsive to the dialogue system output;
processing, at the one or more processors, the additional input data to determine one or more further diagnoses; and
outputting, by the output of the diagnostics system, an indication of the one or more further diagnoses.

23. A diagnostics system, comprising:
one or more processors; and
one or more computer readable media storing computer readable instructions configured to cause the one or more processors to perform a method of automatic diagnosis, the method comprising:
receiving, at an input of a diagnostics system, input data relating to a speech or text input signal originating from a user device, the first input data indicating at least one problem;
processing, at one or more processors, the first input data using a first input pre-processing module comprising a first input pre-processing machine learning model, to generate a representation of the first input data and to generate a first input pre-processing module output based at least in part on the representation of the first input data;
processing, at the one or more processors, the first input pre-processing module output using a preliminary diagnosis machine learning model to determine a preliminary diagnosis output comprising at least one preliminary diagnosis of the problem;
determining, at the one or more processors and based at least in part on the preliminary diagnosis output, at least one dialogue system output;
outputting, by way of an output of the diagnostics system, the dialogue system output;
receiving, at the input of the diagnostics system, additional input data responsive to the dialogue system output;
processing, at the one or more processors, the additional input data to determine one or more further diagnoses; and
outputting, by the output of the diagnostics system, an indication of the one or more further diagnoses.
